(19) United States
(12) Patent Application Publication
(10) Pub. No.: US 2024/0082157 A1
(43) Pub. Date: Mar. 14, 2024

(54) PREPARATION OF LIPOPHILIC ACTIVE INGREDIENTS

(71) Applicant: SPI Pharma, Inc., Wilmington, DE (US)

(72) Inventor: Graeme MACLEOD, Wezembeek Oppem (BE)

(73) Assignee: SPI Pharma, Inc., Wilmington, DE (US)

(21) Appl. No.: 18/261,867

(22) PCT Filed: Jan. 19, 2022
(86) PCT No.: PCT/US22/12944

§ 371 Date: Jul. 18, 2023

Related U.S. Application Data
(60) Provisional application No. 63/139,206, filed on Jan. 19, 2021.

(51) Int. Cl. A61K 9/16; A61K 31/00; A61K 31/216

(52) U.S. Cl. CPC A61K 9/1617; A61K 9/1611; A61K 9/1682; A61K 31/216; A61K 31/658

(57) ABSTRACT

The present disclosure relates to a formulation for a wide variety of poorly soluble drugs to improve bioavailability using a solid self-emulsifying drug delivery system. Compositions of the present disclosure can be used for improved delivery of hydrophobic or lipophilic pharmaceutical active ingredients, such as drugs, nutritional agents, cosmeceuticals, and diagnostic agents.

### CROSS-REFERENCE TO RELATED APPLICATIONS

The present application claims the benefit of priority of U.S. Provisional Patent Application No. 63/139,206 filed on Jan. 19, 2021, which is hereby incorporated by reference in its entirety.

### FIELD OF INVENTION

The present disclosure relates to pharmaceutical delivery systems for pharmaceutical active ingredients, such as drugs, nutritionals, cosmeceuticals, and diagnostic agents. More particularly the compositions of the present disclosure relate to the use of lipids and Hydrophile-Lipophile Balance (HLB) modifying agents to encapsulate or carry one or more active ingredients to prevent first pass metabolism. The present disclosure also relates to a formulation prepared using hot melting process, and/or as part of an additional processing, cooled to a solid lipid particle. The present disclosure also relates to a formulation prepared by placing solid particles into an aqueous liquid to disperse the solid particles into a stable emulsion capable of delivering higher amounts of drug, thereby enhancing dissolution. The present disclosure also relates to compositions comprising a lipophilic active compound, for example a human or veterinary drug or a nutraceutical, with a HLB modifying component.

### BACKGROUND OF THE INVENTION

The therapeutic efficacy of a drug depends upon its bioavailability, which is directly correlated to its solubility. Many drugs, both in development and on the market, are poorly soluble in aqueous media, which can lead to poor bioavailability and frequently results in poor or variable dissolution rates. To achieve the desired drug concentration in systemic circulation to elicit a pharmacological response, solubility is paramount. Different approaches have been taken to achieve a desired level of drug solubility and dissolution rate. These approaches have been based on preparations with increased surface area (micronized powders), molecular inclusion complexes (cyclodextrins and derivatives), co-precipitates with water-soluble polymers (PEG, poloxamers, PVP, HPMC) and non-electrolytes (urea, mannitol, sugars etc.), micellar solutions in surfactant systems (Kolliphor™, Tween™, Gelucires™) and multilayer vesicles (liposomes and niosomes).

Compared to highly soluble compounds, low drug solubility can manifest itself in a variety of unwanted consequences, including (in vivo) decreased bioavailability, an increased chance of a food effect, incomplete release from the dosage form and high inter-patient variability. Despite the formulation challenges, poorly soluble actives are still important class of pharmaceutical compounds for the treatment of a wide range of diseases.

Therefore, there is a need for pharmaceutical compositions and dosage forms to attribute the solid system composed of long chain lipids with a surfactant/solubilizing component that is easily processed and solid/stable at 45-65° C. but which when added to gastric media disperses completely with time and forms an emulsion which then enhances drug dissolution and potentially improves BA by absorption via conventional means or alternatively via the lymphatic system.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BRIEF DESCRIPTION OF THE DRAWINGS

The presently disclosed embodiments will be further explained with reference to the attached drawings. The drawings generally illustrate the principles of the presently disclosed embodiments.

FIGS. 1A-C illustrate (A) a dissolution comparison of Fenofibrate loaded at 2.5, 5, and 10% to raw Fenofibrate powder, (B) a dissolution comparison of Fenofibrate loaded at 20%, 20% with silica, 30%, and 40% to raw Fenofibrate powder, and (C) an electron micrograph of an exemplary bead prepared according to a method of the present disclosure;

FIG. 2 illustrates a dissolution comparison of cannabidiol (CBD) loaded at 2.5, 5, and 10% to raw CBD powder;

FIG. 3 illustrates an example of a liquid triglyceride, or an unsaturated oil;

FIG. 4, comprising FIGS. 4A and 4B, illustrates an example of loading silica with liquid triglyceride, or oil. FIG. 4A is the silica alone (Grace 3150); FIG. 4B is silica with the oil, demonstrating a homogenous mixture;

FIG. 5 illustrates an example of a solid triglyceride (saturated);

FIG. 6, comprising FIGS. 6A, 6B, and 6C, illustrate film casting of fenofibrate active ingredient in solid triglyceride. FIG. 6A is 50 mg/ml fenofibrate in STEROTEX, FIG. 6B is 70 mg/ml fenofibrate in STEROTEX GTP, and FIG. 6C is 50 mg/ml fenofibrate in STEROTEX GTP with 10% TWEEN 80. Film casting is used to determine compatibility of API at different drug loads and the effects of stabilizers, surfactants, and/or co-solvents;

FIG. 7, comprising FIGS. 7A-7D, illustrates compatibility film casting of fenofibrate in solid triglyceride. FIG. 7A is 70 mg/ml fenofibrate in DYNASAN 116, FIG. 7B is 70 mg/ml fenofibrate in STEROTEX GTP (Glyceryl tripalmitate), FIG. 7C is 70 mg/ml fenofibrate in DYNASAN 118, and FIG. 7D is 70 mg/ml fenofibrate in STEROTEX NF (cottonseed oil);

FIG. 8, comprising FIGS. 8A-7D, illustrates compatibility of film casting of fenofibrate in solid triglyceride. FIG. 8A is 70 mg/ml fenofibrate in hydrogenated castor oil (Spectrum), FIG. 8B is 70 mg/ml fenofibrate in STEROTEX K (soybean castor wax), FIG. 8C is 70 mg/ml fenofibrate in STEROTEX HM (soybean oil), and FIG. 8D is 70 mg/ml fenofibrate in cocoa butter;

FIG. 9, comprising FIGS. 9A and 9B, illustrate beads created with DYNASAN 116 (FIG. 9A) and DYNASAN 116 with 50 mg/ml fenofibrate (FIG. 9B) using vibrational drip casting with a Buchi B-390 lab scale spray congealing product; and

FIGS. 10-14 illustrate the spherical nature of lipid particles of the disclosure made by spray coagulation from the formulation of Example 1.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### SUMMARY OF THE INVENTION

In one aspect, the present disclosure relates to a lipid particle comprising, one or more poorly soluble active ingredients; one or more lipids; and/or one or more Hydrophile-Lipophile Balance (HLB) modifying agents to improve a bioavailability of the one or more poorly soluble active ingredients within a subject, wherein the total concentration of the one or more poorly soluble active ingredients in the particle is between about 20% and about 40% by weight. In one embodiment, the one or more lipids is selected from triglycerides, monoglycerides, fatty acids, fluorinated lipids, neutral fats phosphatides, oils, glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myrsitate, glycerol mono-myristate, hydrogenated palm oil, fractionated palm oil, hydrogenated soybean oil, hydrogenated cottonseed oil, hydrogenated castor oil, and steroids. In one embodiment, the one or more lipids comprises a triglyceride. In one embodiment, the triglyceride comprises glycerol tripalmitate. In one embodiment, the glycerol tripalmitate comprises Dynasan 116. In one embodiment, the one or more lipids comprises a monoglyceride. In one embodiment, the monoglyceride is selected from monolaurin, glyceryl monostearate, and glycerol hydroxy stearate. In one embodiment, the monoglyceride comprises glyceryl monostearate. In one embodiment, the glycerol monostearate comprises Imwitor 900K. In one embodiment, the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnanzine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof. In one embodiment, the concentration of the one or more poorly soluble active ingredients in the particle is between about 30% and about 40% by weight. In one embodiment, the lipid particle is a solid lipid particle. In one embodiment, the lipid particle comprises more than one lipid, wherein the total amount of lipid is between about 20% and 80% by weight. In one embodiment, the lipid particle comprises between about 20% and 25% by weight of the one or more poorly soluble active ingredients, between about 18% and 28% by weight of glyceryl monostearate, and between about 30% and 40% by weight of glycerol tripalmitate. In one embodiment, the lipid particle comprises about 20% by weight of the one or more poorly soluble active ingredients, about 23% by weight of glyceryl monostearate, and about 35% of glycerol tripalmitate. In one embodiment, the lipid particle comprises between about 27% to 32% by weight of the one or more poorly soluble active ingredients, between about 15% and 25% by weight of glyceryl monostearate, and between about 25% and 35% of glycerol tripalmitate. In one embodiment, the lipid particle comprises about 30% by weight of the one or more poorly soluble active ingredients, about 20% by weight of glyceryl monostearate, and about 30% of glycerol tripalmitate. In one embodiment, the lipid particle comprises between about 35% and 40% by weight of the one or more poorly soluble active ingredients, between about 12% and 22% by weight of glyceryl monostearate, and between about 21% and 31% of glycerol tripalmitate. In one embodiment, the lipid particle comprises about 40% by weight of the one or more poorly soluble active ingredients, about 17% by weight of glyceryl monostearate, and about 26% of glycerol tripalmitate. In one embodiment, the one or more Hydrophile-Lipophile Balance (HLB) modifying agents in the lipid particle are selected from the group consisting of a surfactant, an emulsifier, lecithin, polyethoxylated castor oil, and Kolliphor EL. In one embodiment, the one or more Hydrophile-Lipophile Balance (HLB) modifying agents in the lipid particle comprise lecithin and polyethoxylated castor oil. In one embodiment, the lipid particle comprises between about 20% and 25% by weight of the one or more poorly soluble active ingredients, between about 7% and 17% by weight of lecithin, and between about 6% and 16% by weight of polyethoxylated castor oil. In one embodiment, the lipid particle comprises about 20% by weight of the one or more poorly soluble active ingredients, about 12% by weight of lecithin, and about 11% by weight of polyethoxylated castor oil. In one embodiment, the lipid particle comprises between about 27% and 32% by weight of the one or more poorly soluble active ingredients, between about 5% and 15% by weight of lecithin, and between about 5% and 15% by weight of polyethoxylated castor oil. In one embodiment, the lipid particle comprises about 30% by weight of the one or more poorly soluble active ingredients, about 10% by weight of lecithin, and about 10% by weight of polyethoxylated castor oil. In one embodiment, the lipid particle comprises between about 35% and 40% by weight of the one or more poorly soluble active ingredients, between about 4% and 14% by weight of lecithin, and between about 3% and 13% by weight of polyethoxylated castor oil. In one embodiment, the lipid particle comprises about 40% by weight of the one or more poorly soluble active ingredients, about 9% by weight of lecithin, and about 8% by weight of polyethoxylated castor oil. In one embodiment, the polyethoxylated castor oil in the lipid particle comprises Kolliphor EL. In one embodiment, the one or more poorly soluble active ingredients comprise fenofibrate. In one embodiment, the one or more poorly soluble active ingredients comprise cannabidiol. In one embodiment, the lipid particle further comprises silica. In one embodiment, a plurality of the lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm; or wherein a plurality of the lipid particles have a size distribution of from about 10 μm to about 500 μm, about 20 μm to about 475 μm, about 30 m to about 450 μm, about 40 μm to about 425 μm, about 50 μm to about 400 μm, about 60 μm to about 375 μm, about 70 μm to about 350 μm, about 80 μm to about 325 μm, about 90 m to about 325 μm, or about 100 μm to about 300 μm. In one embodiment, the particle has a circularity greater than about 0.90. In one embodiment, the particle has a circularity greater than about 0.95. In one embodiment, the particle has an aspect ratio greater than about 0.90. In one embodiment, the particle has an aspect ratio greater than about 0.95.

In another aspect, the present disclosure provides a method of manufacturing a solid lipid particle, wherein the method comprises spray coagulating a lipid particle composition comprising: one or more poorly soluble active ingredients, one or more lipids, and/or one or more Hydrophile-Lipophile Balance (HLB) modifying agents to improve a bioavailability of the one or more poorly soluble active ingredients within a subject. In one embodiment, the solid lipid particle composition further comprises silica. In one embodiment, the method comprises the steps of a) co-melting a first lipid, a second lipid, and a first HLB modifying agent, thereby producing a melt; b) adding a second HLB modifying agent to the melt; c) adding one or more of the poorly soluble active ingredients to the melt; d) co-melting the melt with the second HLB modifying agent and the one or more poorly soluble active ingredients, thereby preparing a mixture; e) transferring the mixture to a heated vessel; f) equilibrating the mixture with the heated vessel, g) spray coagulating the mixture through a spraying chamber to achieve individual droplet separation; and h) cooling the droplets to form solid lipid particles. In one embodiment, c) further comprises adding silica to the melt and the mixture of d) further comprises unmelted silica. In one embodiment, the method further comprises i) coating or co-melting the solid lipid particles with a polymer. In one embodiment, the method further comprises j) preparing a solid suspension of the solid lipid particles with a material having a high melting point. In one embodiment, the first lipid is glyceryl monostearate and the second lipid is glycerol tripalmitate. In one embodiment, the glyceryl monostearate is Imwitor 900K and the glycerol tripalmitate is Dynasan 116. In one embodiment, the first HLB modifying agent is a polyethoxylated castor oil and the second HLB modifying agent is lecithin. In one embodiment, the polyethoxylated castor oil is Kolliphor EL. In one embodiment, the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof. In one embodiment, the one or more poorly soluble active ingredients comprise fenofibrate, cannabidiol, or a combination thereof. In one embodiment, total concentration of the one or more poorly soluble active ingredients in the solid lipid particle is between about 20% and about 40% by weight. In one embodiment, the solid lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm. In one embodiment, solid lipid particle has a circularity greater than about 0.90. In one embodiment, the solid lipid particle has a circularity greater than about 0.95. In one embodiment, the solid lipid particle has an aspect ratio greater than about 0.90. In one embodiment, the solid lipid particle has an aspect ratio greater than about 0.95.

In another aspect, the present disclosure relates to a method of manufacturing a solid lipid particle, wherein the method comprises hot melting a lipid particle composition comprising: one or more poorly soluble active ingredients, one or more lipids, and/or one or more Hydrophile-Lipophile Balance (HLB) modifying agents to improve a bioavailability of the one or more poorly soluble active ingredients within a subject. In one embodiment, the hot melting comprises one or both of hot melt extrusion and hot melt granulation. In one embodiment, the one or more lipids are selected from glyceryl monostearate and glycerol tripalmitate. In one embodiment, the glyceryl monostearate is Imwitor 900K and the glycerol tripalmitate is Dynasan 116. In one embodiment, the lipid particle composition further comprises silica. In one embodiment, the one or more HLB modifying agents are selected from polyethoxylated castor oil and lecithin. In one embodiment, the polyethoxylated castor oil is Kolliphor EL. In one embodiment, the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof. In one embodiment, the one or more poorly soluble active ingredients comprise fenofibrate, cannabidiol, or a combination thereof. In one embodiment, the total concentration of the one or more poorly soluble active ingredients in the solid lipid particle is between about 20% and about 40% by weight. In one embodiment, the solid lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm. In one embodiment, the solid lipid particle has a circularity greater than about 0.90. In one embodiment, the solid lipid particle has a circularity greater than about 0.95. In one embodiment, the solid lipid particle has an aspect ratio greater than about 0.90 In one embodiment, the solid lipid particle has an aspect ratio greater than about 0.95.

In another aspect, the present disclosure relates to a method of manufacturing a solid lipid particle mixture, the method comprising a) co-melting a first lipid, a second lipid, and a first Hydrophile-Lipophile Balance (HLB) modifying agent, thereby producing a melt; b) adding a second HLB modifying agent to the melt; c) adding one or more poorly soluble active ingredients to the melt; d) co-melting the melt with the second HLB modifying agent and the one or more poorly soluble active ingredients, thereby preparing a mixture, e) film casting the mixture, the film casting comprising pouring the mixture and cooling the mixture until hardened, thereby producing the solid lipid particle mixture. In one embodiment, c) further comprises adding silica to the melt and the mixture of d) further comprises unmelted silica. In one embodiment, the method further comprises f) grinding the solid lipid particle mixture into a lipid powder. In one embodiment, the method further comprises g) coating or co-melting the lipid powder with a polymer. In one embodiment, the method further comprises h) preparing a solid suspension of the lipid powder with a material having a high melting point. In one embodiment, the first lipid is glycerol monostearate and the second lipid is glycerol tripalmitate. In one embodiment, the glyceryl monostearate is Imwitor 900K and the glycerol tripalmitate is Dynasan 116. In one embodiment, the first HLB modifying agent is a polyethoxylated castor oil and the second HLB modifying agent is lecithin. In one embodiment, the polyethoxylated castor oil is Kolliphor EL. In one embodiment, the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof. In one embodiment, the one or more poorly soluble active ingredients comprise fenofibrate, cannabidiol, or a combination thereof. In one embodiment, the lipid powder comprises solid lipid particles. In one embodiment, the total concentration of the one or more poorly soluble active ingredients in the solid lipid particle is between about 20% and about 40% by weight. In one embodiment, the solid lipid particles have a size distribution of from about 10 μm to about 500 μm, about 20 μm to about 475 μm, about 30 μm to about 450 μm, about 40 μm to about 425 μm, about 50 μm to about 400 μm, about 60 μm to about 375 μm, about 70 μm to about 350 μm, about 80 μm to about 325 μm, about 90 μm to about 325 μm, or about 100 μm to about 300 μm. In one embodiment, the solid lipid particle has a circularity greater than about 0.90. In one embodiment, the solid lipid particle has a circularity greater than about 0.95. In one embodiment, the solid lipid particle has an aspect ratio greater than about 0.90. In one embodiment, the solid lipid particle has an aspect ratio greater than about 0.95.

In another aspect, the present disclosure relates to a solid dosage form for oral delivery comprising: a) a poorly soluble active ingredient; and b) a solid triglyceride; wherein the poorly soluble active ingredient and the solid triglyceride are spray congealed to form a plurality of beads. In yet another aspect, the present disclosure relates to a solid dosage form for oral delivery comprising a plurality of spray congealed beads, the beads comprising: a) a poorly soluble active ingredient; and b) a solid triglyceride. In one embodiment, the solid triglyceride is selected from DYNASAN® 116, DYNASAN® 118, STEROTEX® GTP, STEROTEX® NF, STEROTEX® K, hydrogenated castor oil, and cocoa butter. In one embodiment, the beads are combined with one or more excipients to form the solid dosage form. In one embodiment, the active ingredient is present in an amount of about 5 mg/mL to about 100 mg/mL, an amount of about 10 mg/mL to about 75 mg/mL, an amount of about 50 mg/mL, or an amount of about 70 mg/mL. In one embodiment, the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof. In one embodiment, the one or more poorly soluble active ingredients comprise fenofibrate, cannabidiol, or a combination thereof. In one embodiment, the beads are lipid particles. In one embodiment, the lipid particles are solid lipid particles. In one embodiment, the total concentration of the one or more poorly soluble active ingredients in the particles is between about 20% and about 40% by weight. In one embodiment, the particles have a size distribution of from about 10 μm to about 500 μm, about 20 μm to about 490 μm, about 30 μm to about 480 μm, about 40 m to about 470 μm, about 50 μm to about 460 μm, about 50 μm to about 450 μm, about 60 m to about 440 μm, about 60 μm to about 430 μm, about 70 μm to about 420 μm, about 70 μm to about 410 μm, or about 75 μm to about 400 μm; or wherein the particles have a size distribution of from about 10 μm to about 500 μm, about 20 μm to about 475 μm, about 30 μm to about 450 μm, about 40 μm to about 425 μm, about 50 μm to about 400 μm, about 60 μm to about 375 μm, about 70 pin to about 350 μm, about 80 μm to about 325 μm, about 90 μm to about 325 μm, or about 100 μm to about 300 μm. In one embodiment, the particles have a circularity greater than about 0.90. In one embodiment, the particles have a circularity greater than about 0.95. In one embodiment, the particles have an aspect ratio greater than about 0.90. In one embodiment, the particles have an aspect ratio greater than about 0.95.

In another aspect, the present disclosure relates to a solid dosage form for oral delivery comprising: a) a poorly soluble active ingredient; b) a liquid triglyceride; and c) silica wherein the poorly soluble active ingredient, the liquid triglyceride, and silica are mixed to form a homogenous mixture. In one embodiment, the liquid triglyceride is selected from sesame oil, olive oil, palm oil, cottonseed oil, corn oil, rapeseed oil, and safflower oil. In one embodiment, the silica is selected from SYLOID® XDP 3150, SYLOID® XDP 3050, ZEOPHARM™ 5191, and ZEOPHARM™ 600. In one embodiment, the homogenous mixture is combined with one or more excipients to form the solid dosage form, solid dosage form. In one embodiment, the active ingredient is present in an amount of about 5 mg/mL to about 100 mg/mL, an amount of about 10 mg/mL to about 75 ng/mL, an amount of about 50 mg/mL, or an amount of about 70 mg/mL. In one embodiment, the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof. In one embodiment, the one or more poorly soluble active ingredients comprise fenofibrate, cannabidiol, or a combination thereof. In one embodiment, the homogenous mixture comprises particles comprising silica loaded with the liquid triglyceride and the poorly soluble active ingredient. In one embodiment, the one or more excipients are blended with particles to form the homogenous mixture, the particles comprising silica loaded with the liquid triglyceride and the poorly soluble active ingredient. In one embodiment, the particles are lipid particles. In one embodiment, the total concentration of the one or more poorly soluble active ingredients in the particles is between about 20% and about 40% by weight. In one embodiment, the particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μn to about 430 μm about 70 μm to about 420 μm, about 70 μm to about 410 μm, or about 75 μm to about 400 μm or wherein the particles have a size distribution of from about 10 μm to about 500 μm, about 20 μm to about 475 μm, about 30 μm to about 450 μm, about 40 m to about 425 μm, about 50 μm to about 400 μm, about 60 μm to about 375 μm, about 70 μm to about 350 μm, about 80 μm to about 325 μm, about 90 μm to about 325 μm, or about 100 μm to about 300 μm. In one embodiment, the particles have a circularity greater than about 0.90. In one embodiment, the particles have a circularity greater than about 0.95. In one embodiment, the particles have an aspect ratio greater than about 0.90 In one embodiment, the particles have an aspect ratio greater than about 0.95.

### DETAILED DESCRIPTION OF THE INVENTION

The present disclosure can provide a number of advantages over conventional methods for the delivery of poorly soluble actives. According to the concept of the present disclosure, the poorly soluble drug is formulated with a lipid and an HLB modifying component.

### Definitions

The term “an active” or “the poorly soluble drug” as used herein interchangeably, refer to an active ingredient that is typically poorly soluble under hydrophilic conditions, including, for example, amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and mixtures thereof.

The term “lipid” as used herein refers to naturally-occurring, synthetic or semi-synthetic (i.e., modified natural) compound which is generally amphipathic. Lipids typically comprise a hydrophilic component and a hydrophobic component. Exemplary lipids include, for example, fatty acids, fluorinated lipids, neutral fats, phosphatides, oils, glycolipids, surface-active agents (surfactants and fluorosurfactants), aliphatic alcohols, waxes, terpenes, triglycerides, diglycerides, monoglycerides, hydrogenated vegetable oils. Some examples are glycerol di-oleate, glycerol mono-oleate, tri-stearin, glycerol di-stearin, glycerol mono-stearin, tri-palmitin, glycerol di-palmate, glycerol mono-palmate, tri-myristinin, glycerol di-myristate, glycerol mono-myristate, hydrogenated palm oil, fractionated palm oil, hydrogenated soybean oil, hydrogenated cottonseed oil, hydrogenated castor oil and mixtures thereof.

The term “monoglycerides” as used herein refers to a molecule derived from glycerol and a single fatty acid, and includes, for example, monolaurin, glyceryl monostearate, glycerol hydroxy stearate and mixtures thereof. Triglycerides are derived from glycerol and three fatty acids. Diglycerides are derived from glycerol and two fatty acids. The term “fatty acid” as used herein refers to a saturated or unsaturated fatty acid, and includes, for example, myristic acid, palmitic acid, stearic acid, oleic acid and mixtures thereof.

HLB (Hydrophile-Lipophile Balance) is an empirical expression for the relationship of the hydrophilic (“water-loving”) and hydrophobic (“water-hating”) groups of a surfactant. The term HLB modifying component includes, for example, a surfactant, emulsifier, and mixtures thereof.

The term “surfactant” as used herein refers to an amphoteric, non-ionic, cationic, or anionic surfactant and includes, for example, sodium lauryl sulphate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, Poloxamer®, Kolliphor EL, Kolliphor RH™, Tween™, Gelucires™ and mixtures thereof. Any surfactant is suitable for use in the present disclosure, whether it be amphoteric, non-ionic, cationic or anionic.

The term “emulsifier” as used herein refers to a substance that stabilizes an emulsion, and includes, for example, egg lecithin, soy lecithin, sodium lauryl sulphate and mixtures thereof.

The term “easily processed” as used herein can refer to a system that is a solid, as opposed to a semi solid, and/or a system that it is not sticky or tacky or a system that has reduced stickiness or tackiness.

The term “high melting point” as used herein can refer to a compound or substance having a melting point of at least 30 degrees Celsius (° C.), at least 35° C., at least 40° C., at least 45° C., at least 50° C., at least 55° C., at least 60° C. at least 65° C. at least 70° C. at least 75° C., at least 80° C., at least 85° C., at least 90° C., at least 95° C., at least 100° C. at least 110° C. at least 120° C., at least 130° C., at least 140° C., or at least 150° C.

The term “effective amount” or “therapeutically effective amount” refers to that amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The specific dose will vary depending on the particular compositions chosen, the dosing regimen to be followed, whether the composition is administered in combination with other compositions or compounds, timing of administration, the tissue to which it is administered, and the physical delivery system in which the composition is carried.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “active pharmaceutical ingredient”, “active ingredient”, “single active”, or “API” may refer to an ingredient that is biologically active. In some cases, the pharmaceutical sample contains one API. In some cases, the pharmaceutical sample contains more than one API.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the present disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but can be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80% 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of an embodiment of the present disclosure. All compositions, methods, and kits described herein can, in alternate embodiments, be more specifically defined by any of the transitional terms “comprising,” “consisting essentially of,” and “consisting of.”

Overview

PCT publication PCT/FR2014/00182 discloses self-emulsifying instant powder system with one or a mixture of cyclodextrins for oral administration. US publication 2005/0209345 discloses lymphatic drug delivery system for lipophilic drugs using combination of lipid and surfactant.

U.S. Pat. No. 6,054,136 discloses microemulsion composition comprising an active along with mixture of fatty acid esters and glycerides, surfactant, co-surfactant and hydrophilic phase. EP Patent No. 1058540 discloses immediate release pellet dosage form to improve the bioavailability of an active. The composition disclosed in EP Patent No. 1058540 is based on Self Micro emulsifying Drug Delivery System wherein it comprises a mixture of one or more active ingredients with a lipophilic phase, a surfactant and a co-surfactant.

U.S. Pat. No. 6,572,892 discloses bead composition for dermatological and cosmetology use comprising hydrophobic wax, oil and talcum. U.S. Pat. No. 7,625,507 discloses the extrusion process for forming chemically stable drug multiparticulates which is intended to embrace a dosage form comprising a multiplicity of particles.

US publication No. 2017/0354599 discloses Lipid multiparticulate formulations with a combination of excipients such as a low flow point excipient, and a high flow point excipient. US publication No. 2014/0357708 discloses cannabidiol composition prepared using self-emulsifying system.

It is an object of the present disclosure to provide an intermediate or a finished formulation of poorly soluble drug. It is a further object of the present disclosure to increase the bioavailability of the formulation of a poorly soluble drug.

The present disclosure also provides a system for the delivery of various active agents which are non-hydrophilic in character within a living body. In another embodiment the present disclosure provides solid lipid particles which forms a solid self-microemulsifying drug delivery system to achieve the increased bioavailability of the poorly soluble drugs.

Thus, in one aspect of the present disclosure, there is provided particles comprising one or more active pharmaceutical ingredient, lipid and/or an HLB modifying agent.

The present disclosure also provides a method of preparing particles comprising one or more active pharmaceutical ingredient, lipid and/or an HLB modifying agent.

Cannabidiol is highly lipophilic, and its oral bioavailability is known to be very low in humans. It is usually supplied via the sublingual route however, it is also available in oil solution, capsule, and nasal spray dosage forms. Cannabidiol is metabolized after administration, and several studies have identified CYP3A4 and CYP2C19 as the major isoforms mediating the metabolism of cannabidiol. Therefore, the oral bioavailability of cannabidiol is affected by both the poor solubility, i.e., low absorption, and the large first-pass effect.

Fenofibrate is hypolipidemic drug used to treat primary hypercholesterolemia, mixed dyslipidemia, and severe hypertriglyceridemia. Fenofibrate is insoluble in water and is rapidly converted to Fenofibric acid (active metabolite) upon oral administration. It is available in the form of tablets and capsules. Like any insoluble drugs. Fenofibrate bioavailability also depends on its solubility.

Fenofibrate is used as a lipid regulating agent and cannabidiol is used for the treatment of epilepsy, anxiety, arthritic pain, sleeping, fibromyalgia, menopause, weight loss, etc. Both of these drugs exhibit poor water solubility and dissolution. Thus, the main object of the present disclosure is to provide the formulation with increased bioavailability for fenofibrate and cannabidiol and drugs with similar physicochemical, pharmacokinetic and pharmacodynamic profiles. The present disclosure also provides the process to prepare fenofibrate and cannabidiol formulation which when placed in the aqueous medium, the solid particles disperse forming a stable emulsion capable of delivering higher amounts of fenofibrate and cannabidiol into solution, thereby enhancing the solubility and consequently the bioavailability of the drug.

The present disclosure also provides the process to prepare fenofibrate and cannabidiol formulations with increased bioavailability.

The present disclosure provides a composition comprising one or more poorly soluble actives, one or more lipids and/or one or more HLB modifying components. In one embodiment, the composition is a lipid particle composition. In one embodiment, a lipid particle made from the composition. Therefore, in one embodiment. the lipid particle comprises the components of the composition. In one embodiment, the lipid particle is a solid lipid particle. In an embodiment, the amount of poorly soluble active component present in the composition is up to 30% of the total weight of the composition. In an embodiment, the active is present in the composition from about 1% to about 30%, from about 5% to about 30%, from about 10% to about 30%, from about 15% to about 30%, from about 20% to about 30%, or from about 25% to about 30 of the total weight of the composition. In another embodiment, the active is present in the composition from about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of the total weight of the composition. In another embodiment, the active is present in the composition from about 20% to about 40% of the total weight of the composition. In one embodiment, the active is present in the composition in about 20%, 21%, 22%, 23%, 24%, 25%, 26%, 27%, 28%, 29%, 30%, 31%, 32%, 33%, 34%, 35%, 36%, 37%, 38%, 39%, or 40% of the total weight of the composition.

In an embodiment of the present disclosure, any hydrophobic active ingredient is useful. In another embodiment, the active is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and mixtures thereof.

In an embodiment of the present disclosure, the active ingredient may be fenofibrate. The active ingredient may be present in an amount of from about 5 mg/ml to about 100 mg/ml, about 10 mg/ml to about 80 mg/ml, about 20 mg/ml to about 75 mg/ml, about 25 mg/ml to about 70 mg/ml, about 30 mg/ml to about 65 mg/ml, about 35 mg/ml to about 60 mg/ml, about 40 mg/ml to about 55 mg/ml, about 45 mg/ml to about 50 mg/ml, about 50 mg/ml, or about 70 mg/ml.

In an embodiment of the present disclosure, the active ingredient is cannabidiol, and is present in an amount of from about 1% to about 10%, from about 5% to about 10%, or at about 5% of the total weight of the final composition.

In another embodiment, the amount of total lipid present in the composition is up to 80% of the total weight of the composition. In an embodiment, more than one lipid is present in the composition. In an embodiment, the total amount of lipid present in the composition is from about 10% to about 80%, from about 15% to about 80%, from about 20% to about 80%, from about 25% to about 80%, from about 30% to about 80%, or from about 35% to about 80%, from about 40% to about 80%, from about 45% to about 80%, from about 50% to about 80%, from about 55% to about 80%, from about 60% to about 80%, from about 65% to about 80%, from about 70% to about 80%, from about 75% to about 80%, or about 80% of the total weight of the composition.

In an embodiment of the present disclosure, the lipid is selected from saturated or unsaturated fatty acids, fluorinated lipids, neutral fats, phosphatides, oils, glycolipids, surface-active agents (surfactants and fluorosurfactants), aliphatic alcohols, waxes, terpenes, triglycerides, diglycerides, monoglycerides, hydrogenated vegetable oils, glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, monolaurin, glyceryl monostearate, glycerol hydroxy stearate, myristic acid, oleic acid, stearic acid, palmitic acid, and mixtures thereof. In an embodiment, the lipid is selected from glyceryl monosterate and glycerol tripalmitate.

In another embodiment of the present disclosure, the HLB modifying component is present in an amount up to 40% of the total weight of the composition. In an embodiment, the total amount of HLB modifying component is present in the composition from about 1% to about 40%, from about 5% to about 40%, from about 10% to about 40%, from about 15% to about 40%, from about 20% to about 40%, or from about 25% to about 40%, from about 30% to about 40%, from about 35% to about 40%, or from about 40% of the total weight of the composition. In an embodiment, the HLB modifying component is selected from one or more of surfactants, emulsifiers, and combinations thereof. In another embodiment, the HLB modifying component is selected from an amphoteric, non-ionic, cationic, or anionic surfactant, sodium lauryl sulphate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, Poloxamer®, Kolliphor EL, Kolliphor RH™, Tween™, Gelucires™, egg lecithin, soy lecithin, sodium lauryl sulphate, and mixtures thereof. In an embodiment, the HLB modifying component is selected from one or more of lecithin and Kolliphor EL.

In an embodiment of the present disclosure, the composition comprises one or more of the lipids glyceryl monostearate (Imwitor 900K) and glycerol tripalmitate (Dynasan 116) and one or more of the HLB modifying components lecithin and Kolliphor EL.

In another embodiment, the present disclosure provides fenofibrate as the active, one or more lipids and/or one or more HLB modifying components. More particularly, the present disclosure provides a fenofibrate composition comprising fenofibrate, glyceryl monostearate (Imwitor 900K), glycerol tripalmitate (Dynasan 116), lecithin and polyethoxylated castor oil (Kolliphor EL).

The present disclosure provides a fenofibrate composition having fenofibrate present up to 30% of the total weight of the composition, glyceryl monostearate (Imwitor 900K) of about 19% of the total weight of the composition, glycerol tripalmitate (Dynasan 116) of about 31% of the total weight of the composition, lecithin of about 10% of the total weight of the composition and polyethoxylated castor oil (Kolliphor EL) of about 10% of the total weight of the composition. In another embodiment, the present disclosure provides fenofibrate composition having an active of about 20% of the total weight of the composition, glyceryl monostearate (Imwitor 900K) of about 22% of the total weight of the composition, glycerol tripalmitate (Dynasan 116) of about 35% of the total weight of the composition, lecithin of about 12% of the total weight of the composition and polyethoxylated castor oil (Kolliphor EL) of about 11% of the total weight of the composition.

In one embodiment, the present disclosure provides cannabidiol as the active, one or more lipids and/or one or more HLB modifying components. More particularly, the present disclosure provides a cannabidiol composition having cannabidiol, glyceryl monostearate (Imwitor 900K), glycerol tripalmitate (Dynasan 116), lecithin and polyethoxylated castor oil (Kolliphor EL).

The present disclosure provides the cannabidiol composition having cannabidiol present up to 30% of the total weight of the composition, glyceryl monostearate (Imwitor 900K) of about 19% of the total weight of the composition, glycerol tripalmitate (Dynasan 116) of about 31% of the total weight of the composition, lecithin of about 10% of the total weight of the composition and polyethoxylated castor oil (Kolliphor EL) of about 10% of the total weight of the composition. In another embodiment, the present disclosure provides cannabidiol composition having an active of about 20% of the total weight of the composition, glyceryl monostearate (Imwitor 900K) of about 22% of the total weight of the composition, glycerol tripalmitate (Dynasan 116) of about 35% of the total weight of the composition, lecithin of about 12% of the total weight of the composition and polyethoxylated castor oil (Kolliphor EL) of about 11% of the total weight of the composition.

The present disclosure also provides the cannabidiol composition having cannabidiol present up to 10% of the total weight of the composition, glyceryl monostearate (Imwitor 900K) of about 25% of the total weight of the composition, glycerol tripalmitate (Dynasan 116) of about 39% of the total weight of the composition, lecithin of about 13% of the total weight of the composition and polyethoxylated castor oil (Kolliphor EL) of about 13% of the total weight of the composition. In another embodiment, the present disclosure provides cannabidiol composition having an active of about 5% of the total weight of the composition, glyceryl monostearate (Imwitor 900K) of about 27% of the total weight of the composition, glycerol tripalmitate (Dynasan 116) of about 41% of the total weight of the composition, lecithin of about 14% of the total weight of the composition and polyethoxylated castor oil (Kolliphor EL) of about 13% of the total weight of the composition.

The present disclosure relates to use of liquid or solid triglycerides in the preparation of an intermediate product comprising a lipophilic active pharmaceutical ingredient (API). Such product may ultimately be used to formulate a dosage form of the API, in particular, an oral dosage form.

In an embodiment of the present disclosure, the intermediate product is comprised of liquid triglycerides and active ingredient, and these components are mixed with silica to create a homogenous mixture. In another embodiment, additional excipients are also included in the mixture to form an oral dosage form such as a sachet or a chewable tablet.

Some examples of liquid triglyceride include sesame oil, olive oil, palm oil, cottonseed oil, corn oil, rapeseed oil, and safflower oil.

Some examples of silica include SYLOID® XDP 3150 (W. R. Grace, Columbia MD), SYLOID® XDP 3050 (W. R. Grace, Columbia, MD), ZEOPHARM™ 5191 (Evonik, Parsipanny, NJ), and ZEOPHARM™ 600 (Evonik, Parsippany, NJ).

In an embodiment of the present disclosure, solid triglycerides and active ingredient are combined to create a solid form to be spray-congealed into beads. Such beads can then be used to formulate a dosage form, such as an oral dosage form. In one embodiment, the beads and optional additional excipients such as Mannogem® EZ or Pharmasperse®, Pharmaburst® 500 (SPI Pharma, Wilmington, DE), magnesium stearate, stabilizers, taste masking agents, or any other excipients typical to an oral dosage formulation, are combined to form an oral dosage form, such as a sachet or chewable tablet.

Some examples of solid triglyceride include DYNASAN® 116, DYNASAN® 118 (IOI Olio GmbH, Germany), STEROTEX® GTP, STEROTEX® NF, STEROTEX® K (Abitec, Columbus, OH), hydrogenated castor oil (Spectrum Chemical, New Brunswick, NJ), and cocoa butter. In one embodiment, DYNASAN® 118 comprises glyceryl tristearate. In one embodiment, DYNASAN® 118 is glyceryl tristearate. In one embodiment, STEROTEX® GTP comprises glycerol tripalmitate. In one embodiment, STEROTEX® GTP is glycerol tripalmitate. In one embodiment, STEROTEX® NF comprises hydrogenated vegetable oil. In one embodiment, STEROTEX® NF is hydrogenated vegetable oil. In one embodiment, STEROTEX® K comprises hydrogenated vegetable oil. In one embodiment, STEROTEX® K is hydrogenated vegetable oil. In one embodiment, STEROTEX® NF and/or STEROTEX® K comprise hydrogenated vegetable oil Type I. In one embodiment, STEROTEX® NF and STEROTEX® K are hydrogenated vegetable oil Type I.

In an embodiment, the lipid particles of the present disclosure are substantially spherical. In one embodiment, the lipid particles have perfect sphericity. “Perfect sphericity” or “perfectly spherical” means a circularity as measured by imaging microscopy of greater than 0.90, and an aspect ratio of less than 1.0. In one embodiment, the particles have a circularity greater than 0.91. In one embodiment, the particles have a circularity greater than 0.92. In one embodiment, the particles have a circularity greater than 0.93. In one embodiment, the particles have a circularity greater than 0.94. In one embodiment, the particles have a circularity greater than 0.95. In one embodiment, the particles have a circularity greater than 0.96. In one embodiment, the particles have a circularity greater than 0.97. In one embodiment, the particles have a circularity greater than 0.98. Circularity is calculated in accordance with International Organization for Standardization (ISO) 9276-6.

Circularity is a measurement of the calculated peripheral length of a circle of the same silhouetted area of a particle's blocking a light source/the particle's actual peripheral length with values in the range from 0-1. A perfect circle has circularity of roundness 1.0, while a needle-shaped object has roundness close to 0. Table 1 show as the circularity of various shapes (Image Analysis Evaluating Particle Shape by Horiba Particles on Jul. 7, 2011 by Jeff Bodycomb www.horiba.com).

TABLE 1
Circularity of various shapes

| Shape | ● | ▪ | ▴ | ▮ | \| | \| |
|---|---|---|---|---|---|---|
| Circularity | 1.0 | 0.886 | 0.777 | 0.660 | 0.509 | 0.4 |

The circularity—is typically determined using the equation Circularity=2(π area)0.5/P; where A is the measured area and P is the perimeter length of the lipid particles. Circularity is calculated in accordance with International Organization for Standardization (ISO) 9276-6 (2008).

In one embodiments, the particles have an aspect ratio of 0.90. In one embodiment, the particles have an aspect ratio of 0.91. In one embodiment, the particles have an aspect ratio of 0.92. In one embodiment, the particles have an aspect ratio of 0.93. In one embodiment, the particles have an aspect ratio of 0.94. In one embodiment, the particles have an aspect ratio of 0.95. In one embodiment, the particles have an aspect ratio of 0.96. In one embodiment, the particles have an aspect ratio of 0.97. In one embodiment, the particles have an aspect ratio of 0.98. In one embodiment, the particles have an aspect ratio of 0.99. In one embodiment, the particles have an aspect ratio of 1.0. In one embodiment, the particles have an aspect ratio of 0.90 or greater. In one embodiment, the particles have an aspect ratio of 0.91 or greater. In one embodiment, the particles have an aspect ratio of 0.92 or greater. In one embodiment, the particles have an aspect ratio of 0.93 or greater. In one embodiment, the particles have an aspect ratio of 0.94 or greater. In one embodiment, the particles have an aspect ratio of 0.95 or greater. In one embodiment, the particles have an aspect ratio of 0.96 or greater. In one embodiment, the particles have an aspect ratio of 0.97 or greater. In one embodiment, the particles have an aspect ratio of 0.98 or greater. In one embodiment, the particles have an aspect ratio of 0.99 or greater. Aspect Ratio is calculated in accordance with International Organization for Standardization (ISO) 9276-6 (2008).

Aspect ratio is defined as the ratio of the length of a sphere divided by the width, with the lipid particles being considered circular (spherical) if the aspect ratio lies between 0.95 and 1.00. Table 2 shows the aspect ratios of various shapes (Image Analysis: Evaluating Particle Shape by Horiba Particles on Jul. 7, 2011 by Jeff Bodycomb www.horiba.com). The aspect ratio is sensitive to how isometric the particle is. Particles with a high aspect ratio not only tumble but they tend to lodge in pore spaces in the coating bed and bounce as they tumble. This is a measure of rod, plate or needle-like characteristics of a particle.

TABLE 2
Aspect ratio of various shapes

| Shape | ● | ▪ | ▴ | ▮ | \| | \| |
|---|---|---|---|---|---|---|
| Circularity | 1.0 | 0.886 | 0.777 | 0.660 | 0.509 | 0.4 |
| Aspect Factor | 1.0 | 1.0 | 1.0 | 0.25 | 0.10 | 0.05 |
| Aspect Ratio | 1:1 | 1:1 | 1:1 | 1:4 | 1:10 | 1:20 |

Aspect ratio is the ratio of the shortest diameter of particle to the longest diameter of a particle. Aspect ratio is calculated in accordance with International Organization for Standardization (ISO) 9276-6 (2008). Feret measured diameters as parallel lines brought in to touch particle at any angle. Thus it is the shortest separation of these lines divide by the longest separation.

In an embodiment, the lipid particles have a solidity ratio of 0.99. In one embodiment, 30% of the lipid particles have a solidity ratio of 0.99. In one embodiment, 40% of the lipid particles have a solidity ratio of 0.99. In one embodiment, 50% of the lipid particles have a solidity ratio of 0.99. In one embodiment, 60% of the lipid particles have a solidity ratio of 0.99. In one embodiment, 70% of the lipid particles have a solidity ratio of 0.99. In one embodiment, 80% of the lipid particles have a solidity ratio of 0.99. In one embodiment, 90% of the lipid particles have a solidity ratio of 0.99. In one embodiment, 30% or more of the lipid particles have a solidity ratio of 0.99. In one embodiment, 40% or more of the lipid particles have a solidity ratio of 0.99. In one embodiment, 50% or more of the lipid particles have a solidity ratio of 0.99. In one embodiment, 60% or more of the lipid particles have a solidity ratio of 0.99. In one embodiment, 70% or more of the lipid particles have a solidity ratio of 0.99. In one embodiment, 80% or more of the lipid particles have a solidity ratio of 0.99. In one embodiment, 90% or more of the lipid particles have a solidity ratio of 0.99. Solidity Ratio is calculated in accordance with International Organization for Standardization (ISO) 9276-6 (2008).

Solidity looks for missing areas caused by risers or indentations in the surface of the microsphere or particle. In order to determine the solidity, a cord is wrapped around the particle to approximate the area of the particle without convex (indented areas) due to crevices and risers off the surface. The area of the particle is exactly measured as the shadow of the image of the particle in a light path. The area of the particle is then divided by the area inside the cord stretched over the particles' outer surface. Solidity is calculated in accordance with International Organization for Standardization (ISO) 9276-6 (2008).

A sphere has a solidity of 1. A cube, a triangle (pyramid) or a rod would also have a solidity of 1. Although a cube and a pyramid have corners/edges, they do not have surface risers or crevices. Any surface indentations or surface bumps would add to the area inside the cord. Thus solidity as a factor is then related to the area associated with the convexity area of the particle as area lost ratio to solidity.

In an embodiment, the lipid particles have a convexity ratio of 0.99. In one embodiment, 30% of the lipid particles have a convexity ratio of 0.99. In one embodiment, 40% of the lipid particles have a convexity ratio of 0.99. In one embodiment, 50% of the lipid particles have a convexity ratio of 0.99. In one embodiment, 60% of the lipid particles have a convexity ratio of 0.99. In one embodiment, 70% of the lipid particles have a convexity ratio of 0.99. In one embodiment, 80% of the lipid particles have a convexity ratio of 0.99. In one embodiment, 30% or more of the lipid particles have a convexity ratio of 0.99. In one embodiment, 40% or more of the lipid particles have a convexity ratio of 0.99. In one embodiment, 50% or more of the lipid particles have a convexity ratio of 0.99. In one embodiment, 60% or more of the lipid particles have a convexity ratio of 0.99. In one embodiment, 70% or more of the lipid particles have a convexity ratio of 0.99. In one embodiment, 80% or more of the lipid particles have a convexity ratio of 0.99. Convexity Ratio is calculated in accordance with International Organization for Standardization (ISO) 9276-6 (2008).

Convexity is similar to solidity but focuses more on surface smoothness. Here the most accurate measurement is the periphery of the particle. What the approximation is in this index is the cord length that is drawn surrounding the particle which is the same cord length as in the solidity measurement. If the surface is perfectly smooth and without crevices or risers the convexity will be equal to one. Convexity is calculated in accordance with International Organization for Standardization (ISO) 9276-6 (2008).

In an embodiment, the lipid particles are microspheres. In one embodiment, the microspheres have a mean particle size from about 2 μm to about 1500 μm. In one embodiment, the microspheres have a mean particle size from about 10 μm to about 500 μm. In one embodiment, the microspheres have a mean particle size from about 25 μm to about 475 μm, about 50 μm to about 450 μm, about 50 μm to about 425 μm, or about 75 μm to about 400 μm. In one embodiment, the microspheres have a particle size distribution of from about 10 μm to about 500 μm, about 25 μm to about 475 μm, about 50 μm to about 450 μm, about 50 μm to about 425 μm, or about 75 μm to about 400 μm. In another embodiment, the microspheres have a mean particle size from about 25 μm to about 500 μm, about 50 μm to about 450 μm, about 50 μm to about 400 μm, about 75 μm to about 350 μm, or about 100 μm to about 300 μm. In one embodiment, the microspheres have a particle size distribution of from about 25 μm to about 500 μm, about 50 μm to about 450 μm, about 50 μm to about 400 μm, about 75 μm to about 350 μm, or about 100 μm to about 300 μm.

In an embodiment, a microsphere or lipid particle of the present disclosure has a particle size distribution (d(0.9)/d(0.1)) of 2.8 or less. In one embodiment, the microsphere or lipid particle has a particle size distribution of 2.7 or less, 2.4 or less, 2.3 or less, 2.2 or less, 2.1 or less, 2.0 or less, 1.9 or less, or 1.8 or less.

The present disclosure also provides a process to prepare lipid particle compositions for improving the bioavailability of poorly soluble drugs using a spray coagulation method, film casting method, hot melt extrusion or hot melt granulation method

Spray Coagulation Method for Creating Solid Lipid Particles

In this method, molten lipid is sprayed into a cooling chamber and on contact with the cool air, congeals into spherical solid particles. The parameters to be considered when preparing a composition according to the present disclosure are the melting point of the excipients, the viscosity of the formulation and the cooling air temperature inside the chamber to allow instant solidification of the droplets. In this application, the spray coagulation method is used to prepare the solid lipid particles. The method is as described below:
a. Glyceryl monostearate (Imwitor 900K), glycerol tripalmitate (Dynasan 116) and polyethoxylated castor oil (Kolliphor EL) are co-melted in 50 ml beaker at 140° C. until the melting process is completed.
b. Once fully melted, lecithin is added to the melt and stirred at 200 rpm.
c. The mixture is then transferred to a heated syringe and allowed to equilibrate to 100° C.
d. The solid lipid mixture is then spray congealed through a Buchi B-390.
e. The mixture is pumped into the heat block, which contains the vibratory atomizer
f. The nozzle size is set at 80 μm opening and the vibratory atomizer frequency and amplitude are configured to achieve individual droplet separation.
g. Droplets are cooled through a residence time within a cylinder designed to cool ambient air to approximately −6° C.
h. Solid lipid particles are then collected at the bottom of this chamber at a particle size distribution (PSD) between 75-400 μm

Film Casting Method for Crating Solid Lipid Particles

The Film casting method is a predictive tool used in pre-formulation setting. In this method, a drug-lipid film is cast on glass plates and later milled to a desired particle size. The method used in this application is described as below:
a. Glyceryl monostearate (Imwitor 900K), glycerol tripalmitate (Dynasan 116) and polyethoxylated castor oil (Kolliphor EL) are co-melted in 50 ml beaker at 140° C. until the melting process is completed.
b. Once fully melted, lecithin is then added to the melt and stirred at 200 rpm until the lecithin was fully melted.
c. The mixture is then poured onto a room temperature flat surface until the mixture hardened.
d. The solid lipid mixture is then ground until a powder at around 100-300 μm was obtained.

The lipid particles obtained with the processes as described above are further formulated into the final dosage forms such as tablet, sachet, stick packs, capsule, etc., using techniques known in the art. The final dosage form is prepared using inactive excipients like lubricant, glidant, carrier, flavours, sweeteners etc. Lubricants useful in the present disclosure include magnesium stearate, sodium stearyl fumarate, talc, silica, boric acid, sodium benzoate, sodium oleate, sodium acetate, sodium lauryl sulphate, magnesium lauryl sulphate, sodium stearate, magnesium stearate, wax and mixtures thereof. Glidants useful in the present disclosure include magnesium stearate, colloidal silicon dioxide, silica gel, starch, talc and mixtures thereof. Sweeteners used in the present disclosure include sucrose, glucose, glycerol, sucralose, sorbitol, saccharin sodium, aspartame and mixtures thereof. Flavouring agents useful in the present disclosure include orange, chocolate, mint, fruit flavours and mixtures thereof. Carrier systems or diluents useful in the present disclosure include sugar or carbohydrate or polyol-based materials such as mannitol, erythritol, sucrose, Commercial examples of such carrier systems include Mannogem EZ or Pharmasperse 415 and mixtures thereof.

In another embodiment, the tackiness or the stickiness of the lipid particle formulation of the present disclosure can be reduced by adding an additional material to the formulation, by making the lipid particle formulation using a less tacky material, or by increasing the solidity of the material, as described above. It is contemplated that by reducing the tackiness or stickiness of the lipid particle formulation, the resultant particles can be more stable when packaged together (e.g., reducing aggregation of the particles in the package) or stored together. This concept is further expanded upon below (see, e.g., Suspension of solid).

Particle Coating

Once particles are created through either method described above, particles can be coated with titanium dioxide, silicon dioxide, mannitol, lactose, calcium silicate, magnesium stearate, and starch to reduce particle to particle connections that cause agglomeration.

Suspension of a Solid

A material with a high melting point can be added to the lipid mixture once the lipid mixture prepared above is melted (e.g., to reduce or eliminate the tackiness or stickiness of the lipid particle formulation). In certain embodiments, about 5% to about 50% by weight of a material with a high melting point can be added to the lipid mixture. In certain embodiments, about 5%, about 10%, about 15%, about 20%, about 25%, about 30%, about 35%, about 40%, about 45%, or about 50% by weight of a material with a high melting point can be added to the lipid mixture. The material added is required to remain solid, where the addition of a soluble or insoluble material can significantly increase the viscosity of the material to further solidify the material. The addition of a solid material can also act as pore former or swelling agents to increase disintegration time of the solid lipid particles where the solid material is also soluble. Examples of pore formers or swelling agents include materials such as sugars (e.g. sucrose, glucose, maltose), polyols (e.g. mannitol, maltitol, lactitol), and hydrophilic polymers (e.g. PVP, PVA, HPMC, PEG 400-800, sodium starch glycolate, or i-HPC).

Co-Melting with a Polymer

Polymers with moderate to high melting points like PEG 1500-8000, Poly vinyl alcohol, and Polox can be co-melted with the lipid system to add stability to the particles and also act to increase the wetting or disintegration time of the particles.

In one embodiment, the lipid particles prepared using spray coagulation method or film casting method can be further coated to improve their processability or functionality. The coating of the lipid particles can be done using the method as described below:

500 g of the lipid particles can be loaded into a lab scale fluid bed processor such as a GPCG 1.1 Glatt coater using a Wurster column and bottom spraying approach. A solution of HPMC can be prepared by dissolving the HPMC E15 grade in water at around 10% weight loading. The resultant solution can be applied to the pellets by fluidizing them in air and applying the coating via the nozzle and atomizing the solution. The product temperature during the coating operation would be around 40° C. An alternative coating material to apply to the multiparticulates could be ethylcellulose which comes as a latex suspension in the form of Aquacoat or Surelease. The Aquacoat would be plasticized via the addition of 22% (calculated as a % of the solids in the dispersion) of a plasticizer such as triethyl citrate stirred under low agitation for around 30 mins. The Aquacoat or Surelease systems could be diluted by the addition of water prior to use by adding up to 40% w/w water. The same process approach could be undertaken to apply the ethylcellulose based systems. The total polymer weight loading applied would be around 3-10% expressed as a % of the multiparticulate weight and depending on the polymer system used. Other polymers that could be applied to the multiparticulates include polymethacrylates, cellulose acetates, hydroxypropyl methylcellulose phthalate, HPMCAS (hydroxypropyl-methylcellulose acetate succinate), polyvinyl alcohol Commercial examples of these types of polymers that are pre-formulated for ease of use include Opadry, Eudragit, Kollicoat and Methocel. Polymer coatings could also be applied by using powder layering techniques.

The following clauses describe certain embodiments of the present disclosure:

Clause 1—A lipid particle composition, comprising: one or more poorly soluble active ingredients; one or more lipids; and/or one or more Hydrophile-Lipophile Balance (HLB) modifying agents to improve a bioavailability of the one or more poorly soluble active ingredients within a subject.

Clause 2—The lipid particle composition of clause 1, wherein the one or more lipids and/or the one or more HLB modifying agents comprise a solid system, and wherein the solid system is easily processed and/or stable at a temperature ranging from between about 45° C. and about 65° C.

Clause 3—The lipid particle composition of clause 2, wherein the one or more lipids comprise long chain lipids.

Clause 4—The lipid particle composition of any one of clauses 1-3, wherein the one or more poorly soluble active ingredients is selected from the group consisting of amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and any combination thereof.

Clause 5—The lipid particle composition of clause 4, wherein the one or more poorly soluble active ingredients is selected from the group consisting of fenofibrate and cannabidiol.

Clause 6—The lipid particle composition of any one of clauses 1-5, wherein the one or more lipids is selected from the group consisting of triglycerides, fatty acids, fluorinated lipids, neutral fats, phosphatides, oils, glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, and steroids.

Clause 7—The lipid particle composition of clause 6 wherein the one or more lipids comprises a triglyceride.

Clause 8—The lipid particle composition of clause 6 or 7, wherein the triglyceride is derived from one or both of glycerol and fatty acids.

Clause 9—The lipid particle composition of any one of clauses 6-8, wherein the triglyceride comprises glyceryl tripalmitate.

Clause 10—The lipid particle composition of any one of clauses 1-9, wherein the one or more lipids comprises a monoglyceride.

Clause 11—The lipid particle composition of clause 10, wherein the monoglyceride is selected from the group consisting of monolaurin, glyceryl monostearate, and glycerol hydroxy stearate.

Clause 12—The lipid particle composition of clause 11, wherein monoglyceride comprises glyceryl monostearate.

Clause 13—The lipid particle composition of any one of clauses 1-12, wherein the one or more lipids comprises a diglyceride.

Clause 14—The lipid particle composition of any one of clauses 1-13, wherein the one or more HLB modifying agents is selected from the group consisting of a surfactant, and an emulsifier.

Clause 15—The lipid particle composition of clause 14 comprising the surfactant, wherein the surfactant is selected from the group consisting of sodium lauryl sulphate, monooleate, monolaurate, monopalmitate, monostearate, an ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer, Kolliphor EL, Tween™, and Gelucires™.

Clause 16—The lipid particle composition of clause 14 or 15 comprising the emulsifier, wherein the emulsifier is selected from the group consisting of egg lecithin, soy lecithin, and sodium lauryl sulphate.

Clause 17—The lipid particle composition of any one of clauses 1-16, wherein the one or more poorly soluble active ingredients comprises at most about 10%, at most about 20%, at most about 30%, or at most about 40% by weight of the lipid particle composition.

Clause 18—The lipid particle composition of any one of clauses 1-17, wherein the one or more HLB modifying agents comprises at most about 40% by weight of the lipid particle composition.

Clause 19—The lipid particle composition of any one of clauses 1-18, wherein the one or more lipids comprises at most about 80% by weight of the lipid particle composition.

Clause 20—A method of manufacturing the solid lipid particle composition of any one of clauses 1-19, wherein the method comprises spray coagulating at least two of the one or more poorly soluble active ingredients, the one or more lipids, and the one or more Hydrophile-Lipophile Balance (HLB) modifying agents.

Clause 21—A method of manufacturing the solid lipid particle composition of any one of clauses 1-19, wherein the method comprises film casting at least two of the one or more poorly soluble active ingredients, the one or more lipids, and the one or more Hydrophile-Lipophile Balance (HLB) modifying agents.

Clause 22—A method of manufacturing the solid lipid particle composition of any one of clauses 1-19, wherein the method comprises hot melting at least two of the one or more poorly soluble active ingredients, the one or more lipids, and the one or more Hydrophile-Lipophile Balance (HLB) modifying agents.

Clause 23—The method of clause 22, wherein the hot melting comprises one or both of hot melt extrusion and hot melt granulation.

Clause 24—A cannabidiol lipid particle composition, comprising: one or more cannabidiols; one or more lipids; and/or one or more Hydrophile-Lipophile Balance (HLB) modifying agents to improve a bioavailability of the one or more poorly soluble active ingredients within a subject.

Clause 25—The cannabidiol lipid particle composition of clause 24, wherein the one or more lipids and/or the one or more HLB modifying agents comprise a solid system, and wherein the solid system is easily processed and/or stable at a temperature ranging from between about 45° C. and about 65° C.

Clause 26—The cannabidiol lipid particle composition of clause 24 or 25, wherein the one or more lipids is selected from group consisting of triglycerides, fatty acids, fluorinated lipids, neutral fats, phosphatides, oils, glycolipids, surface-active agents, surfactants, fluorosurfactants, aliphatic alcohols, waxes, terpenes, triglycerides, diglycerides, monoglycerides, hydrogenated vegetable oils, glycerol di-oleate, glycerol mono-oleate, tri-stearin, glycerol di-stearin, glycerol mono-stearin, tri-palmitin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, and steroids.

Clause 27—The cannabidiol lipid particle composition of clause 26, wherein the one or more lipids comprises a triglyceride.

Clause 28—The cannabidiol lipid particle composition of clause 26 or 27, wherein the triglyceride comprises glyceryl tripalmitate.

Clause 29—The cannabidiol lipid particle composition of any one of clauses 24-28, wherein the one or more lipids comprises monoglyceride.

Clause 30—The cannabidiol lipid particle composition of clause 29, wherein the monoglyceride comprises glyceryl monostearate.

Clause 31—The cannabidiol lipid particle composition of any one of clauses 24-30, wherein the one or more lipids comprises diglyceride.

Clause 32—The cannabidiol lipid particle composition of any one of clauses 24-31, wherein the one or more HLB modifying agents is selected from the group consisting of a surfactant and an emulsifier.

Clause 33—The cannabidiol lipid particle composition of clause 32 comprising the surfactant, wherein the surfactant comprises Kolliphor EL.

Clause 34—The cannabidiol lipid particle composition of clause 32 or 33 comprising the emulsifier, wherein the emulsifier is selected from the group consisting of egg lecithin, soy lecithin, and sodium lauryl sulphate.

Clause 35—A cannabidiol lipid particle composition, comprising: one or more cannabidiols; a glyceryl monostearate (Imwitor 900K); a glycerol tripalmitate (Dynasan 116), and a polyethoxylated castor oil (Kolliphor EL).

Clause 36—The cannabidiol lipid particle composition of clause 35, wherein the one or more cannabidiols comprises about 20% by weight of the cannabidiol lipid particle composition.

Clause 37—The cannabidiol lipid particle composition of clause 35 or 36, wherein the glyceryl monostearate (Imwitor 900K) comprises about 22% by weight of the cannabidiol lipid particle composition.

Clause 38—The cannabidiol lipid particle composition of any one of clauses 35-37, wherein the glycerol tripalmitate (Dynasan 116) comprises about 35% by weight of the cannabidiol lipid particle composition.

Clause 39—The cannabidiol lipid particle composition of any one of clauses 35-38, wherein the polyethoxylated castor oil (Kolliphor EL) comprises about 11% by weight of the cannabidiol lipid particle composition.

Clause 40—The cannabidiol lipid particle composition of any one of clauses 35-39, further comprising lecithin.

Clause 41—The cannabidiol lipid particle composition of clause 40, wherein the lecithin comprises about 12% by weight of the cannabidiol lipid particle composition.

Clause 42—The cannabidiol lipid particle composition of clause 35, wherein the one or more cannabidiols comprises about 10% by weight of the cannabidiol lipid particle composition.

Clause 43—The cannabidiol lipid particle composition of clause 35 or 42, wherein the glyceryl monostearate (Imwitor 900K) comprises about 25% by weight of the cannabidiol lipid particle composition.

Clause 44—The cannabidiol lipid particle composition of any one of clauses 35, 42, and 43, wherein the glycerol tripalmitate (Dynasan 116) comprises about 39% by weight of the cannabidiol lipid particle composition.

Clause 45—The cannabidiol lipid particle composition of any one of clauses 35, and 4244, wherein the polyethoxylated castor oil (Kolliphor EL) comprises about 13% by weight of the cannabidiol lipid particle composition.

Clause 46—The cannabidiol lipid particle composition of any one of clauses 35, and 4245, further comprising lecithin.

Clause 47—The cannabidiol lipid particle composition of clause 46, wherein the lecithin comprises about 13% by weight of the cannabidiol lipid particle composition.

Clause 48—The cannabidiol lipid particle composition of clause 35, wherein the one or more cannabidiols comprises about 5% by weight of the cannabidiol lipid particle composition.

Clause 49—The cannabidiol lipid particle composition of clause 35 or 48, wherein the glyceryl monostearate (Imwitor 900K) comprises about 27% by weight of the cannabidiol lipid particle composition.

Clause 50—The cannabidiol lipid particle composition of any one of clauses 35, 48, and 49, wherein the glycerol tripalmitate (Dynasan 116) comprises about 41% by weight of the cannabidiol lipid particle composition.

Clause 51—The cannabidiol lipid particle composition of any one of clauses 35, and 48-50, wherein the polyethoxylated castor oil (Kolliphor EL) comprises about 13% by weight of the cannabidiol lipid particle composition.

Clause 52—The cannabidiol lipid particle composition of any one of clauses 35, and 48-51, further comprising lecithin.

Clause 53—The cannabidiol lipid particle composition of clause 52, wherein the lecithin comprises about 14% by weight of the cannabidiol lipid particle composition.

Clause 54—A method of manufacturing a cannabidiol lipid particle composition using spray coagulation, the method comprising: (a) co-melting Imwitor 900K, Dynasan 116, and Kolliphor EL, thereby producing a melt; (b) adding lecithin to the melt; (c) adding a cannabidiol (CBD) to the melt; (d) co-melting the melt with the lecithin and the CBD, thereby preparing a mixture; (e) transferring the mixture to a heated vessel; (f) equilibrating the mixture with the heated vessel; (g) spray coagulating the mixture through a spraying chamber to achieve individual droplet separation; and (h) cooling of the droplets to form solid lipid particles.

Clause 55—The method of clause 54, further comprising collecting the solid lipid particles.

Clause 56—The method of clause 54 or clause 55, further comprising coating or co-melting the solid lipid particles with a polymer.

Clause 57—The method of clause 54 or clause 55, further comprising preparing a solid suspension of the solid lipid particles with a material having a high melting point.

Clause 58—A method of manufacturing a cannabidiol lipid particle composition using film casting, the method comprising: (a) co-melting Imwitor 900K, Dynasan 116, and Kolliphor EL, thereby producing a melt; (b) adding lecithin to the melt; (c) adding cannabidiol (CBD) to the melt; (d) co-melting the melt with the lecithin and the CBD, thereby preparing a mixture; (e) film casting the mixture, the film casting comprising pouring the mixture and cooling the mixture until hardened, thereby producing a solid lipid mixture.

Clause 59—The method of clause 58, further comprising grinding the solid lipid mixture into a lipid powder.

Clause 60—The method of clause 58 or 59, further comprising coating or co-melting the lipid powder with a polymer.

Clause 61—The method of clause 58 or 59, further comprising preparing a solid suspension of the lipid powder with a material having a high melting point.

Clause 62—A method of preparing a cannabidiol sachet or a stick pack, the method comprising: (a) mixing solid lipid pellets of cannabidiol with a glidants; (b) adding a sweetener, thereby producing a mixture; and (c) packaging the mixture in a sachet or a stick pack.

Clause 63—A method of preparing a cannabidiol sachet or a stick pack, the method comprising: (a) mixing solid lipid pellets of cannabidiol with a magnesium stearate; (b) adding a fruit flavour and/or a sucralose, thereby producing a mixture; and (c) packaging the mixture in a sachet or a stick pack.

Clause 64—A method of preparing a cannabidiol sachet or a stick pack, the method comprising: (a) adding a carrier system comprising Mannogem EZ or Pharmasperse 415 with solid lipid particles of cannabidiol, thereby producing a first mixture; (b) adding a magnesium stearate to the first mixture, thereby producing a second mixture; (c) adding a fruit flavour and/or a sucralose to the second mixture, and (d) packaging the mixture in a sachet or a stick pack.

Clause 65—A method of preparing a cannabidiol tablet composition, the method comprising: (a) mixing the cannabidiol lipid particle composition of any one of clauses 24-61 with at least one excipient or lubricant, thereby producing a blend; and (b) compressing the blend into a tablet.

Clause 66—A method of preparing a cannabidiol tablet composition, the method comprising: (a) mixing the cannabidiol lipid particle composition of any one of clauses 24-61 with one or both of mannitol and magnesium stearate, thereby producing a blend; and (b) compressing the blend into a tablet.

Clause 67—A solid dosage form for oral delivery comprising: (a) an active ingredient; and (b) a solid triglyceride; wherein said active ingredient and solid triglyceride are spray congealed to form a bead.

Clause 68—A solid dosage form for oral delivery comprising: (a) an active ingredient; (b) a liquid triglyceride; and (c) a silica, wherein said active ingredient, liquid triglyceride, and silica are mixed to form a homogenous mixture.

Clause 69—The solid dosage form of clause 67 or 68, wherein the active ingredient is lipophilic.

Clause 70—The solid dosage form of clause 69, wherein the active ingredient is fenofibrate.

Clause 71—The solid dosage form of 67, wherein the solid triglyceride is selected from the group consisting of DYNASAN® 116, DYNASAN® 118, STEROTEX® GTP, STEROTEX® NF, STEROTEX® K, hydrogenated castor oil, and cocoa butter.

Clause 72—The solid dosage form of clause 68, wherein the liquid triglyceride is selected from the group consisting of sesame oil, olive oil, palm oil, cottonseed oil, corn oil, rapeseed oil, and safflower oil.

Clause 73—The solid dosage form of clause 68, wherein the silica is selected from the group consisting of SYLOID® XDP 3150, SYLOID® XDP 3050, ZEOPHARM™ 5191, and ZEOPHARM™ 600.

Clause 74—The solid dosage form of clause 67 or 68, wherein the active ingredient is present in an amount of about 5 mg/ml to about 100 mg/ml.

Clause 75—The solid dosage form of clause 74, wherein the active ingredient is present in an amount of about 10 mg/ml to about 75 mg/ml.

Clause 76—The solid dosage form of clause 75, wherein the active ingredient is present in an amount of about 50 mg/ml.

Clause 77—The solid dosage form of clause 75, wherein the active ingredient is present in an amount of about 70 mg/ml.

Clause 78—The solid dosage form of clause 67, further wherein said bead is combined with one or more other excipients to form a solid dosage form.

Clause 79—The solid dosage form of clause 68, further wherein said mixture is combined with one or more other excipients to form a solid dosage form.

Clause 80—The lipid particle composition of any one of clauses 1-19, wherein the one or more lipids are selected from the group consisting of a glyceryl monostearate, and a glycerol tripalmitate.

Clause 81—The lipid particle composition of clause 80, wherein the one or more lipids comprises the glyceryl monostearate and the glycerol tripalmitate.

Clause 82—The lipid particle composition of clause 81, comprising about 20% by weight of the one or more poorly soluble active ingredients, about 23% by weight of the glyceryl monostearate, and about 35% of the glycerol tripalmitate.

Clause 83—The lipid particle composition of clause 81, comprising about 30% by weight of the one or more poorly soluble active ingredients, about 20% by weight of the glyceryl monostearate, and about 30% of the glycerol tripalmitate.

Clause 84—The lipid particle composition of clause 81, comprising about 40% by weight of the one or more poorly soluble active ingredients, about 17% by weight of the glyceryl monostearate, and about 26% of the glycerol tripalmitate.

Clause 85—The lipid particle composition of any one of clauses 80-84, wherein the glycerol monostearate comprises Imwitor 900K.

Clause 86—The lipid particle composition of any one of clauses 80-85, wherein the glycerol tripalmitate comprises Dynasan 116.

Clause 87—The lipid particle composition of any one of clauses 1-19, and 80-86 wherein the one or more Hydrophile-Lipophile Balance (HLB) modifying agents are selected from the group consisting of a surfactant, an emulsifier, lecithin and, polyethoxylated castor oil, and Kolliphor EL.

Clause 88—The lipid particle composition of any one of clauses 1-19, and 80-86 wherein the one or more Hydrophile-Lipophile Balance (HLB) modifying agents comprise lecithin and polyethoxylated castor oil.

Clause 89—The lipid particle composition of clause 88, comprising about 20% by weight of the one or more poorly soluble active ingredients, about 12% by weight of the lecithin, and about 11% of the polyethoxylated castor oil.

Clause 90—The lipid particle composition of clause 88, comprising about 30% by weight of the one or more poorly soluble active ingredients, about 10% by weight of the lecithin, and about 10% of the polyethoxylated castor oil.

Clause 91—The lipid particle composition of clause 88, comprising about 40% by weight of the one or more poorly soluble active ingredients, about 9% by weight of the lecithin, and about 8% of the polyethoxylated castor oil.

Clause 92—The lipid particle composition of any one of clauses 88-91, wherein the polyethoxylated castor oil comprises Kolliphor EL.

Clause 93—The lipid particle composition of any one of clauses 80-92, wherein the one or more poorly soluble active ingredients comprise fenofibrate.

Clause 201—A lipid particle comprising: one or more poorly soluble active ingredients; one or more lipids; and/or one or more Hydrophile-Lipophile Balance (HLB) modifying agents to improve a bioavailability of the one or more poorly soluble active ingredients within a subject, wherein the total concentration of the one or more poorly soluble active ingredients in the particle is between about 20% and about 40% by weight.

Clause 202—The lipid particle of clause 201, wherein the one or more lipids is selected from triglycerides, monoglycerides, fatty acids, fluorinated lipids, neutral fats, phosphatides, oils, glycerol di-oleate, glycerol mono-oleate, tri-stearin, glycerol di-stearin, glycerol mono-stearin, tri-palmintin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, and steroids, comprises the glyceryl monostearate and the glycerol tripalmitate.

Clause 203—The lipid particle of clause 202, wherein the one or more lipids comprises a triglyceride.

Clause 204—The lipid particle of clause 203, wherein the triglyceride comprises glycerol tripalmitate.

Clause 205—The lipid particle of clause 204, wherein the glycerol tripalmitate comprises Dynasan 116.

Clause 206—The lipid particle of any one of clauses 202-205, wherein the one or more lipids comprises a monoglyceride.

Clause 207—The lipid particle of clause 206, wherein the monoglyceride is selected from monolaurin, glyceryl monostearate, and glycerol hydroxy stearate.

Clause 208—The lipid particle of clause 205, wherein the monoglyceride comprises glyceryl monostearate.

Clause 209—The lipid particle of clause 208, wherein the glycerol monostearate comprises Imwitor 900K.

Clause 210—The lipid particle of any one of clauses 201-209, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

Clause 211—The lipid particle of any one of clauses 201-210, wherein the concentration of the one or more poorly soluble active ingredients in the particle is between about 30% and about 40% by weight.

Clause 212—The lipid particle of any one of clauses 201-211, wherein the lipid particle is a solid lipid particle.

Clause 213—The lipid particle of any one of clauses 201-212, comprising more than one lipid, wherein the total amount of lipid is between about 20% and 80% by weight.

Clause 214—The lipid particle of any one of clauses 201-213, comprising between about 20% and 25% by weight of the one or more poorly soluble active ingredients, between about 18% and 28% by weight of glyceryl monostearate, and between about 30% and 40% by weight of glycerol tripalmitate.

Clause 215—The lipid particle of clause 214, comprising about 20% by weight of the one or more poorly soluble active ingredients, about 23% by weight of glyceryl monostearate, and about 35% of glycerol tripalmitate.

Clause 216—The lipid particle of any one of clauses 201-213, comprising between about 27% to 32% by weight of the one or more poorly soluble active ingredients, between about 15% and 25% by weight of glyceryl monostearate, and between about 25% and 35% of glycerol tripalmitate.

Clause 217—The lipid particle of clause 216, comprising about 30% by weight of the one or more poorly soluble active ingredients, about 20% by weight of glyceryl monostearate, and about 30% of glycerol tripalmitate.

Clause 218—The lipid particle of any one of clauses 201-213, comprising between about 35% and 40% by weight of the one or more poorly soluble active ingredients, between about 12% and 22% by weight of glyceryl monostearate, and between about 21% and 31% of glycerol tripalmitate.

Clause 219—The lipid particle of clause 218, comprising about 40% by weight of the one or more poorly soluble active ingredients, about 17% by weight of glyceryl monostearate, and about 26% of glycerol tripalmitate.

Clause 220—The lipid particle of any one of clauses 201-219, wherein the one or more Hydrophile-Lipophile Balance (HLB) modifying agents are selected from the group consisting of a surfactant, an emulsifier, lecithin, polyethoxylated castor oil, and Kolliphor EL.

Clause 221—The lipid particle of any one of clauses 201-219, wherein the one or more Hydrophile-Lipophile Balance (HLB) modifying agents comprise lecithin and polyethoxylated castor oil.

Clause 222—The lipid particle of clause 220 or clause 221, comprising between about 20% and 25% by weight of the one or more poorly soluble active ingredients, between about 7% and 17% by weight of lecithin, and between about 6% and 16% by weight of polyethoxylated castor oil.

Clause 223—The lipid particle of clause 222, comprising about 20% by weight of the one or more poorly soluble active ingredients, about 12% by weight of lecithin, and about 11% by weight of polyethoxylated castor oil.

Clause 224—The lipid particle of clause 220 or clause 221, comprising between about 27% and 32% by weight of the one or more poorly soluble active ingredients, between about 5% and 15% by weight of lecithin, and between about 5% and 15% by weight of polyethoxylated castor oil.

Clause 225—The lipid particle of clause 224, comprising about 30% by weight of the one or more poorly soluble active ingredients, about 10% by weight of lecithin, and about 10% by weight of polyethoxylated castor oil.

Clause 226—The lipid particle of clause 220 or clause 221, comprising between about 35% and 40% by weight of the one or more poorly soluble active ingredients, between about 4% and 14% by weight of lecithin, and between about 3% and 13% by weight of polyethoxylated castor oil.

Clause 227—The lipid particle of clause 226, comprising about 40% by weight of the one or more poorly soluble active ingredients, about 9% by weight of lecithin, and about 8% by weight of polyethoxylated castor oil.

Clause 228—The lipid particle of any one of clauses 221-227, wherein the polyethoxylated castor oil comprises Kolliphor EL.

Clause 229—The lipid particle of any one of clauses 201-228, wherein the one or more poorly soluble active ingredients comprise fenofibrate.

Clause 230—The lipid particle of any one of clauses 201-229, wherein the one or more poorly soluble active ingredients comprise cannabidiol.

Clause 231—The lipid particle of any one of clauses 201-230, further comprising silica.

Clause 232—The lipid particle of any one of clauses 201-231, wherein a plurality of the lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm; or wherein a plurality of the lipid particles have a size distribution of from about 10 μm to about 500 μm, about 20 μm to about 475 μm, about 30 μm to about 450 μm, about 40 μm to about 425 μm, about 50 μm to about 400 μm, about 60 μm to about 375 μm, about 70 μm to about 350 μm, about 80 μm to about 325 μm, about 90 μm to about 325 μm, or about 100 μm to about 300 μm.

Clause 233—The lipid particle of any one of clauses 201-232, wherein the particle has a circularity greater than about 0.90.

Clause 234—The lipid particle of any one of clauses 201-233, wherein the particle has a circularity greater than about 0.95.

Clause 235—The lipid particle of any one of clauses 201-234, wherein the particle has an aspect ratio greater than about 0.90.

Clause 236—The lipid particle of any one of clauses 201-235, wherein the particle has an aspect ratio greater than about 0.95.

Clause 237—A method of manufacturing a solid lipid particle, wherein the method comprises spray coagulating a lipid particle composition comprising: one or more poorly soluble active ingredients, one or more lipids, and/or one or more Hydrophile-Lipophile Balance (HLB) modifying agents to improve a bioavailability of the one or more poorly soluble active ingredients within a subject.

Clause 238—The method of clause 237, wherein the solid lipid particle composition further comprises silica.

Clause 239—The method of clause 237, comprising the steps of: a) co-melting a first lipid, a second lipid, and a first HLB modifying agent, thereby producing a melt; b) adding a second HLB modifying agent to the melt; c) adding one or more of the poorly soluble active ingredients to the melt; d) co-melting the melt with the second HLB modifying agent and the one or more poorly soluble active ingredients, thereby preparing a mixture; e) transferring the mixture to a heated vessel; f) equilibrating the mixture with the heated vessel; g) spray coagulating the mixture through a spraying chamber to achieve individual droplet separation; and h) cooling the droplets to form solid lipid particles.

Clause 240—The method of clause 239, wherein c) further comprises adding silica to the melt and the mixture of d) further comprises unmelted silica.

Clause 241—The method of clause 239 or clause 240, further comprising i) coating or co-melting the solid lipid particles with a polymer.

Clause 242—The method of any one of clauses 239-241, further comprising j) preparing a solid suspension of the solid lipid particles with a material having a high melting point.

Clause 243—The method of any one of clauses 239-242, wherein the first lipid is glyceryl monostearate and the second lipid is glycerol tripalmitate.

Clause 244—The method of clause 243, wherein the glyceryl monostearate is Imwitor 900K and the glycerol tripalmitate is Dynasan 116.

Clause 245—The method of any one of clauses 239-244, wherein the first HLB modifying agent is a polyethoxylated castor oil and the second HLB modifying agent is lecithin.

Clause 246—The method of clause 245, wherein the polyethoxylated castor oil is Kolliphor EL.

Clause 247—The method of any one of clauses 237-246, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

Clause 248—The method of any one of clauses 237-247, wherein the one or more poorly soluble active ingredients comprise fenofibrate, cannabidiol, or a combination thereof.

Clause 249—The method of any one of clauses 237-248, wherein the total concentration of the one or more poorly soluble active ingredients in the solid lipid particle is between about 20% and about 40% by weight.

Clause 250—The method of any one of clauses 237-249, wherein the solid lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm.

Clause 251—The method of any one of clauses 237-250, wherein the solid lipid particle has a circularity greater than about 0.90.

Clause 252—The method of any one of clauses 237-251, wherein the solid lipid particle has a circularity greater than about 0.95.

Clause 253—The method of any one of clauses 237-252, wherein the solid lipid particle has an aspect ratio greater than about 0.90.

Clause 254—The method of any one of clauses 237-253, wherein the solid lipid particle has an aspect ratio greater than about 0.95.

Clause 255—A method of manufacturing a solid lipid particle, wherein the method comprises hot melting a lipid particle composition comprising: one or more poorly soluble active ingredients, one or more lipids, and/or one or more Hydrophile-Lipophile Balance (HLB) modifying agents to improve a bioavailability of the one or more poorly soluble active ingredients within a subject.

Clause 256—The method of clause 255, wherein the hot melting comprises one or both of hot melt extrusion and hot melt granulation.

Clause 257—The method of clause 255 or clause 256, wherein the one or more lipids are selected from glyceryl monostearate and glycerol tripalmitate.

Clause 258—The method of clause 257, wherein the glyceryl monostearate is Imwitor 900K and the glycerol tripalmitate is Dynasan 116.

Clause 259—The method of any one of clauses 255-258, wherein the lipid particle composition further comprises silica.

Clause 260—The method of any one of clauses 255-259, wherein the one or more HLB modifying agents are selected from polyethoxylated castor oil and lecithin.

Clause 261—The method of clause 260, wherein the polyethoxylated castor oil is Kolliphor EL.

Clause 262—The method of any one of clauses 255-261, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

Clause 263—The method of any one of clauses 255-262, wherein the one or more poorly soluble active ingredients comprise fenofibrate, cannabidiol, or a combination thereof.

Clause 264—The method of any one of clauses 255-263, wherein the total concentration of the one or more poorly soluble active ingredients in the solid lipid particle is between about 20% and about 40% by weight.

Clause 265—The method of any one of clauses 255-264, wherein the solid lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm.

Clause 266—The method of any one of clauses 255-265, wherein the solid lipid particle has a circularity greater than about 0.90.

Clause 267—The method of any one of clauses 255-266, wherein the solid lipid particle has a circularity greater than about 0.95.

Clause 268—The method of any one of clauses 255-267, wherein the solid lipid particle has an aspect ratio greater than about 0.90.

Clause 269—The method of any one of clauses 255-268, wherein the solid lipid particle has an aspect ratio greater than about 0.95.

Clause 270—A method of manufacturing a solid lipid particle mixture, the method comprising: a) co-melting a first lipid, a second lipid, and a first Hydrophile-Lipophile Balance (HLB) modifying agent, thereby producing a melt; b) adding a second HLB modifying agent to the melt; c) adding one or more poorly soluble active ingredients to the melt; d) co-melting the melt with the second HLB modifying agent and the one or more poorly soluble active ingredients, thereby preparing a mixture; e) film casting the mixture, the film casting comprising pouring the mixture and cooling the mixture until hardened, thereby producing the solid lipid particle mixture.

Clause 271—The method of clause 270, wherein c) further comprises adding silica to the melt and the mixture of d) further comprises unmelted silica.

Clause 272—The method of clause 270 or clause 271, further comprising f) grinding the solid lipid particle mixture into a lipid powder.

Clause 273—The method of clause 272, further comprising g) coating or co-melting the lipid powder with a polymer.

Clause 274—The method of clause 272 or clause 273, further comprising h) preparing a solid suspension of the lipid powder with a material having a high melting point.

Clause 275—The method of any one of clauses 270-274, wherein the first lipid is glyceryl monostearate and the second lipid is glycerol tripalmitate.

Clause 276—The method of clause 275, wherein the glyceryl monostearate is Imwitor 900K and the glycerol tripalmitate is Dynasan 116.

Clause 277—The method of any one of clauses 270-276, wherein the first HLB modifying agent is a polyethoxylated castor oil and the second HLB modifying agent is lecithin.

Clause 278—The method of clause 277, wherein the polyethoxylated castor oil is Kolliphor EL.

Clause 279—The method of any one of clauses 270-278, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

Clause 280—The method of any one of clauses 270-279, wherein the one or more poorly soluble active ingredients comprise fenofibrate, cannabidiol, or a combination thereof.

Clause 281—The method of clause 272 or clause 273 wherein the lipid powder comprises solid lipid particles.

Clause 282—The method of clause 281, wherein the total concentration of the one or more poorly soluble active ingredients in the solid lipid particle is between about 20% and about 40% by weight.

Clause 283—The method of clause 281 or clause 282, wherein the solid lipid particles have a size distribution of from about 10 μm to about 500 μm, about 20 μm to about 475 μm, about 30 μm to about 450 μm, about 40 μm to about 425 μm, about 50 μm to about 400 μm, about 60 μm to about 375 μm, about 70 μm to about 350 μm, about 80 μm to about 325 μm, about 90 μm to about 325 μm, or about 100 μm to about 300 μm.

Clause 284—The method of any one of clauses 281-283, wherein the solid lipid particle has a circularity greater than about 0.90.

Clause 285—The method of any one of clauses 281-284, wherein the solid lipid particle has a circularity greater than about 0.95.

Clause 286—The method of any one of clauses 281-285, wherein the solid lipid particle has an aspect ratio greater than about 0.90.

Clause 287—The method of any one of clauses 281-286, wherein the solid lipid particle has an aspect ratio greater than about 0.95.

Clause 288—A solid dosage form for oral delivery comprising: a) a poorly soluble active ingredient; and b) a solid triglyceride; wherein the poorly soluble active ingredient and the solid triglyceride are spray congealed to form a plurality of beads.

Clause 289—A solid dosage form for oral delivery comprising a plurality of spray congealed beads, the beads comprising: a) a poorly soluble active ingredient; and b) a solid triglyceride.

Clause 290—The solid dosage form of clause 288 or clause 289, wherein the solid triglyceride is selected from DYNASAN® 116, DYNASAN® 118, STEROTEX® GTP, STEROTEX® NF, STEROTEX® K, hydrogenated castor oil, and cocoa butter.

Clause 291—The solid dosage form of any one of clauses 288-290, wherein the beads are combined with one or more excipients to form the solid dosage form.

Clause 292—A solid dosage form for oral delivery comprising: a) a poorly soluble active ingredient; b) a liquid triglyceride; and c) silica wherein the poorly soluble active ingredient, the liquid triglyceride, and silica are mixed to form a homogenous mixture.

Clause 293—The solid dosage form of clause 292, wherein the liquid triglyceride is selected from sesame oil, olive oil, palm oil, cottonseed oil, corn oil, rapeseed oil, and safflower oil.

Clause 294—The solid dosage form of clause 292 or clause 293, wherein the silica is selected from SYLOID® XDP 3150, SYLOID® XDP 3050, ZEOPHARM™ 5191, and ZEOPHARM™ 600.

Clause 295—The solid dosage form of any one of clauses 292-294, wherein the homogenous mixture is combined with one or more excipients to form the solid dosage form.

Clause 296—The solid dosage form of any one of clauses 288-295, wherein the active ingredient is present in an amount of about 5 mg/mL to about 100 mg/mL, an amount of about 10 mg/mL to about 75 mg/mL, an amount of about 50 mg/mL, or an amount of about 70 mg/mL.

Clause 297—The solid dosage form of any one of clauses 288-2%, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

Clause 298—The solid dosage form of any one of clauses 288-297, wherein the one or more poorly soluble active ingredients comprise fenofibrate, cannabidiol, or a combination thereof.

Clause 299—The solid dosage form of any one of clauses 288-291, wherein the beads are lipid particles.

Clause 300—The solid dosage form of clause 299, wherein the lipid particles are solid lipid particles.

Clause 301—The solid dosage form of any one of clauses 292-295, wherein the homogenous mixture comprises particles comprising silica loaded with the liquid triglyceride and the poorly soluble active ingredient.

Clause 302—The solid dosage form of clause 295, wherein the one or more excipients are blended with particles to form the homogenous mixture, the particles comprising silica loaded with the liquid triglyceride and the poorly soluble active ingredient.

Clause 303—The solid dosage form of clause 301 or clause 302, wherein the particles are lipid particles.

Clause 304—The solid dosage form of any one of clauses 299-303, wherein the total concentration of the one or more poorly soluble active ingredients in the particles is between about 20% and about 40% by weight.

Clause 305—The solid dosage form of any one of clauses 299-304, wherein the particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm; or wherein the particles have a size distribution of from about 10 μm to about 500 μm, about 20 μm to about 475 μm, about 30 μm to about 450 μm, about 40 μm to about 425 μm, about 50 μm to about 400 μm, about 60 μm to about 375 μm, about 70 μm to about 350 μm, about 80 μm to about 325 μm, about 90 μm to about 325 μm, or about 100 μm to about 300 μm.

Clause 306—The solid dosage form of any one of clauses 299-305, wherein the particles have a circularity greater than about 0.90.

Clause 307—The solid dosage form of any one of clauses 299-306, wherein the particles have a circularity greater than about 0.95.

Clause 308—The solid dosage form of any one of clauses 299-307, wherein the particles have an aspect ratio greater than about 0.90.

Clause 309—The solid dosage form of any one of clauses 299-308, wherein the particles have an aspect ratio greater than about 0.95.

The present disclosure is further explained with the following examples. The example is demonstrated by using cannabidiol (CBD) as an active wherein solid lipid particles of CBD are prepared and later used in preparing a final dosage form e. g. sachet, stick pack, orally dispersible powder, orally dispersible tablet, lozenge, chewable or swallow tablets.

### EXAMPLES

### Example 1—Preparation of Solid Lipid Particles of Cannabidiol

| | Percentage of |
|---|---|
| Component | formulation |
| Imwitor 900K | 27% |
| Dynasan 116 | 41% |
| Lecithin | 14% |
| Kolliphor EL | 13% |
| CBD | 5% |
| Total | 100% |

Detailed Method of Preparation:

Method 1—Spray Coagulation Method for Creating Solid Lipid Particles of Cannabidiol

Imwitor 900K, Dynasan 116, and Kolliphor EL were co-melted in a 50 ml beaker at 140° C. Once fully melted, lecithin was added to the melt and stirred at 200 RPM until lecithin completely melted. Cannabidiol (CBD) was then added and co-melted in the mixture. The mixture was further transferred to a heated syringe and allowed to equilibrate to 100° C. The solid lipid mixture was then spray coagulated through a Buchi B-390 and the mixture was pumped into the heat block having the vibratory atomizer.

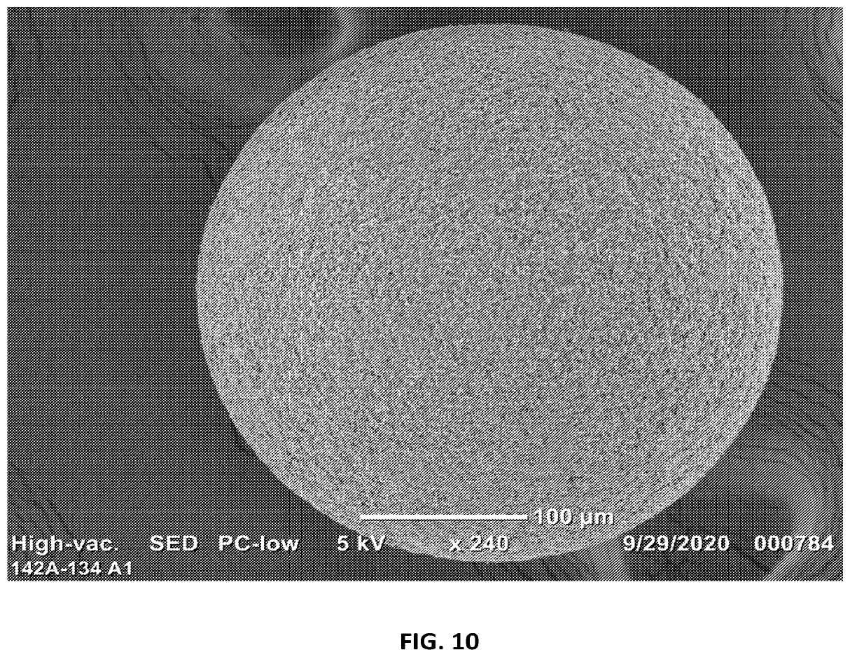

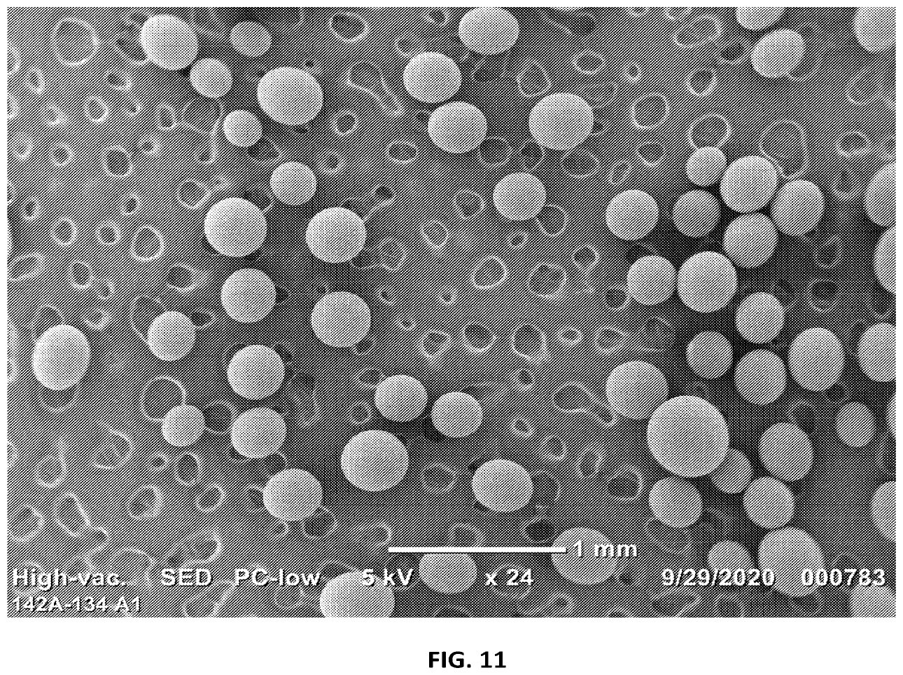

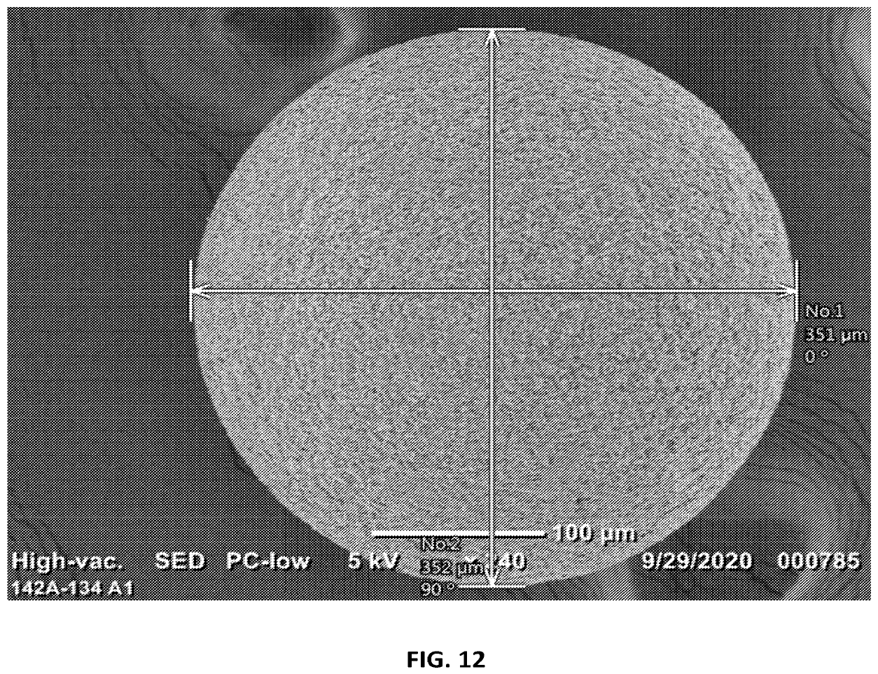

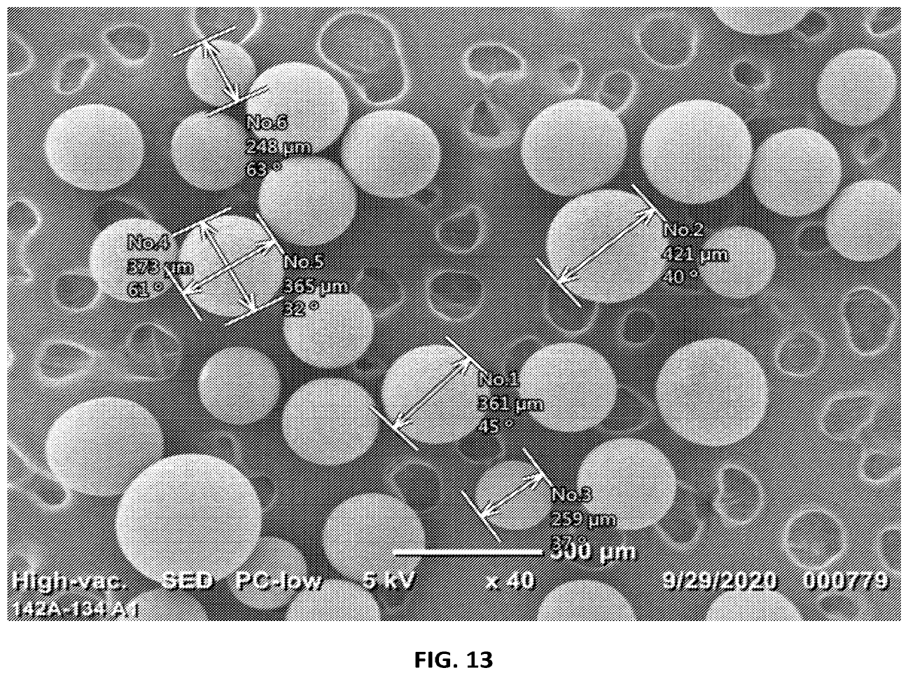

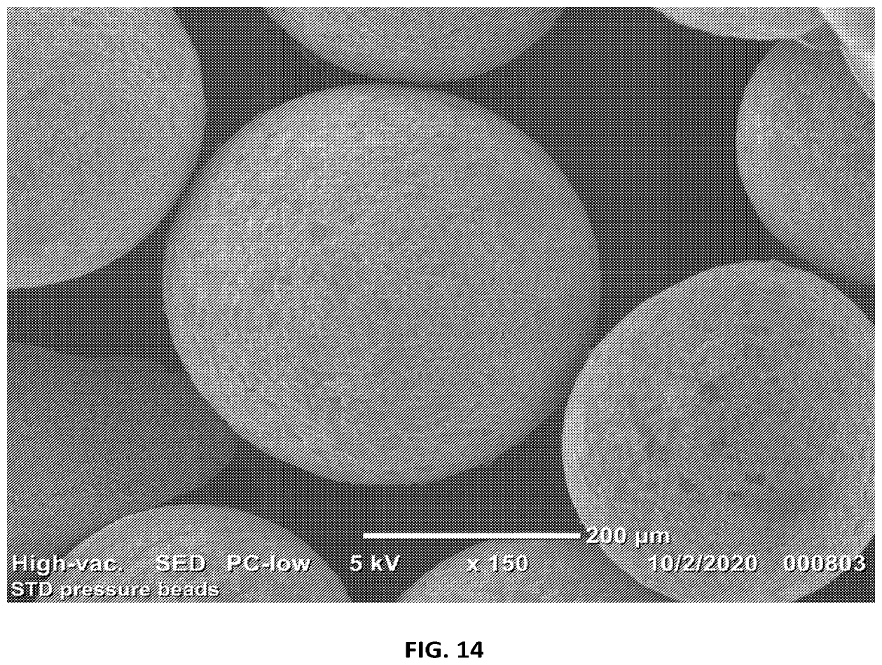

The nozzle size was set at 80 μm opening and the vibratory atomizer frequency and amplitude were configured to achieve individual droplet separation Droplets were further cooled through a residence time within a cylinder designed to cool ambient are to ˜−6° C. Solid lipid particles were then collected at the bottom of this chamber at a PSD between 150-200 μm. FIGS. 10-14 depict the sphericity of solid lipid particles made via spray coagulation of the above formulation

Method 2—Film Casting Method for Creating Solid Lipid Particles of Cannabidiol

Imwitor 900K. Dynasan 116, and Kolliphor EL were co-melted in a 50 ml beaker at 140° C. Once fully melted, lecithin was added to the melt and stirred at 200 RPM until lecithin until lecithin completely melted. Cannabidiol (CBD) was then added and co-melted in the mixture. The mixture was then poured onto a flat surface at room temperature and was allowed to cool until hardened. The solid lipid mixture, was then ground until a powder at around 100-300 μm obtained.

Solid lipid pellets derived through either spray coagulation or film casting can be used in orally dispersible powder formulations or in tablets prepared using direct compression process.

Sachet and Stickpacks of Cannabidiol

A. Using Solid Lipid Particles Only

90-98% of Solid lipid pellets comprising an active are mixed with magnesium stearate at a level of about 1-5% for 5 minutes. 0.25-1% of fruit flavour and 0.1-1.5% of sucralose are further added to the mixture and the mixture is filled into an aluminium sachet using manual filling or an automated stick pack filling machine and packed as sachets or as stick packs.

The sachet or a stick pack prepared according to a method of the present disclosure is further explained with the following example:

| Component | Percentage |
|---|---|
| Solid lipid pellet of | 96.75% |
| CBD | |
| Magnesium stearate | 2% |
| Fruit flavour | 0.75% |
| Sucralose | 0.5% |
| Total | 100% |

B. Using Solid Lipid Particles with a Carrier

Mannogem EZ (a spray dried mannitol) or other carriers like Pharmasperse 415 can be added to assist as a flow agent and carrier. About 5-40% of the soluble carrier system is added to 56.75-92.25% of the solid lipid particles and mixed for 5-15 minutes. Magnesium stearate of about 2% to act as a glidant is added to the mixture for 5-10 minutes 0.25-1% of fruit flavour and 0.1-1.5% of sucralose are further added to the mixture. The mixture is then filled into an aluminium sachet using manual filling or an automated stick pack filling machine and added by weight to the desired dosage.

The sachet or a stick pack prepared with carrier system according a method of the present disclosure is further explained with the following example:

| Component | Percentage |
|---|---|
| Solid lipid pellet of CBD | 72.5% |
| Carrier (Mannogem EZ or Pharmasperse | 24.25% |
| 415) | |
| Magnesium stearate | 2% |
| Fruit flavour | 0.75% |
| Sucralose | 0.5% |
| Total | 100% |

Tablets of Cannabidiol

0.1-80% of the Solid lipid particles of CBD of about are blended with 20-75% of diluent and 0.1-3% of lubricant. The obtained mixture is then compressed into a tablet.

The tablet prepared according a method of the present disclosure is further explained with the following example:

| Component | Percentage |
|---|---|
| Solid lipid particles of CBD | 75% |
| Diluent [Mannitol (Mannogem XL)] | 24.75% |
| Lubricant (Mg stearate or sodium stearyl | 0.25% |
| fumarate) | |
| Total | 100% |

The following reference examples are further offered to illustrate, but not to limit, the present disclosure to embodiments provided in the reference examples.

### Reference Example 1

| | Percentage of |
|---|---|
| Component | formulation |
| Imwitor 900K | 27.8% |
| Dynasan 116 | 40.7% |
| Lecithin | 13.1% |
| Kolliphor EL | 12.4% |
| Progesterone | 6.0% |
| Total | 100% |

### Reference Example 2

| | Percentage of |
|---|---|
| Component | formulation |
| Imwitor 900K | 25.8% |
| Dynasan 116 | 38.8% |
| Lecithin | 13.0% |
| Kolliphor EL | 12.4% |
| Docusate | 5.0% |
| Cyclosporin | 5.0% |
| Total | 100% |

### Reference Example 3

| | Percentage of |
|---|---|
| Component | formulation |
| Imwitor 900K | 25.8% |
| Dynasan 116 | 38.8% |
| Lecithin | 13.0% |
| Kolliphor EL | 12.4% |
| Furosemide | 10.0% |
| Total | 100% |

### Reference Example 4

| | Percentage of |
|---|---|
| Component | formulation |
| Imwitor 900K | 25.8% |
| Dynasan 116 | 38.8% |
| Lecithin | 13.0% |
| Kolliphor EL | 12.4% |
| Docusate | 5.0% |
| Desmopressin | 5.0% |
| Total | 100% |

In another embodiment, the present disclosure also teaches preferred silica formulation used in a formulations of the present disclosure.

Docusate and Propyl Gallate

The use of 12% docusate showed the fastest release of CBD with a low change in the release profile over 1 month at 40° C. 75% RH. As known, docusate is not commonly used in these formulations. Therefore, the findings show that docusate or ionic surfactants are strong candidates for immediate release in silica formulations.

| | Percentage of |
|---|---|
| Component | formulation |
| Sesame Oil | 32% |
| CBD | 9% |
| Syloid 3150 | 46% |
| Docusate | 12% |
| Propyl gallate | 1% |
| Total | 100% |

PEG and Propyl gallate

The Use of 12% PEG 400 Showed a Moderate Release of CBD with a Very Low Change in the release profile over 1 month at 40° C., 75% RH.

| | Percentage of |
|---|---|
| Component | formulation |
| Sesame Oil | 32% |
| CBD | 0% |
| Syloid 3150 | 46% |
| PEG 400 | 12% |
| Propyl gallate | 1% |
| Total | 100% |

Docusate+Kolliphor EL+Propyl Gallate

The use of 6% Kolliphor EL and 6% Docusate showed a moderate release of CBD with the lowest change in the release profile over 1 month at 40° C., 75% RH.

| | Percentage of |
|---|---|
| Component | formulation |
| Sesame Oil | 32% |
| CBD | 9% |
| Syloid 3150 | 46% |
| Kolliphor EL | 6% |
| Docusate | 6% |
| Propyl gallate | 1% |
| Total | 100% |

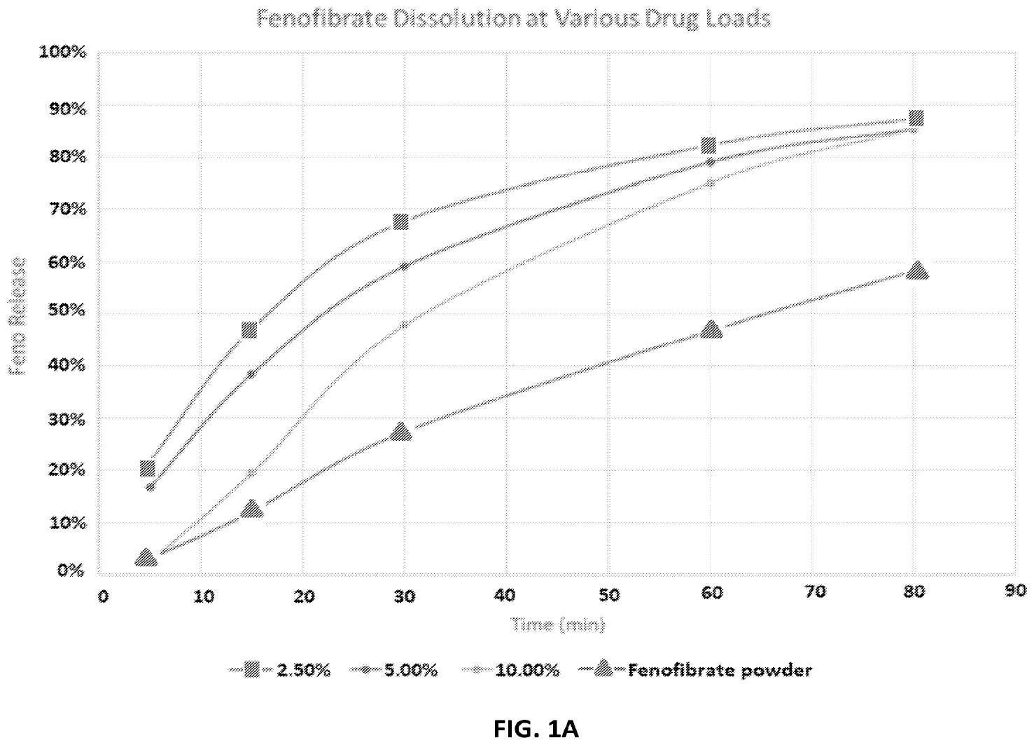

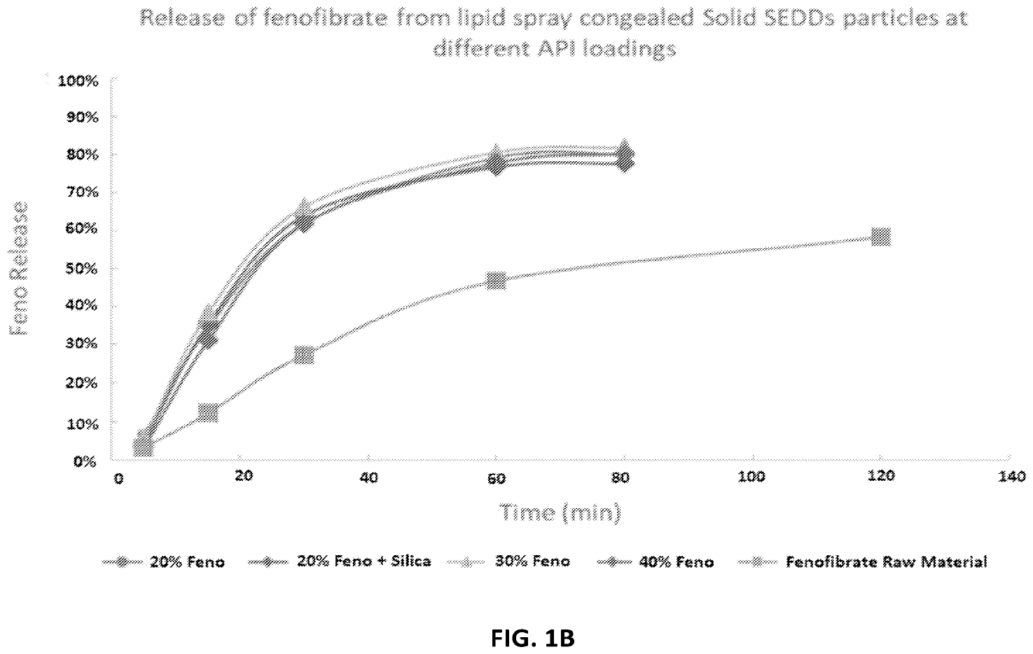

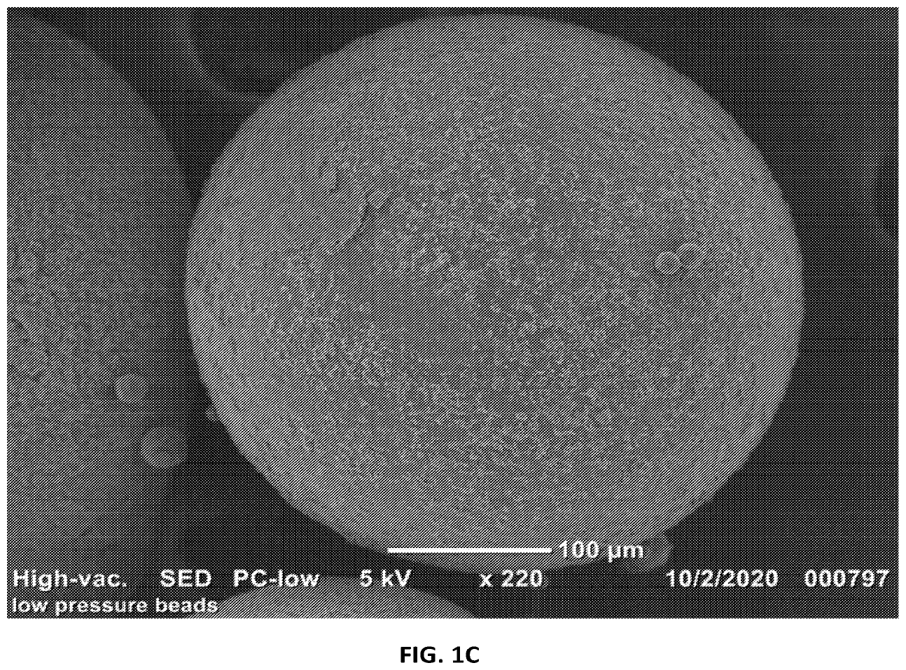

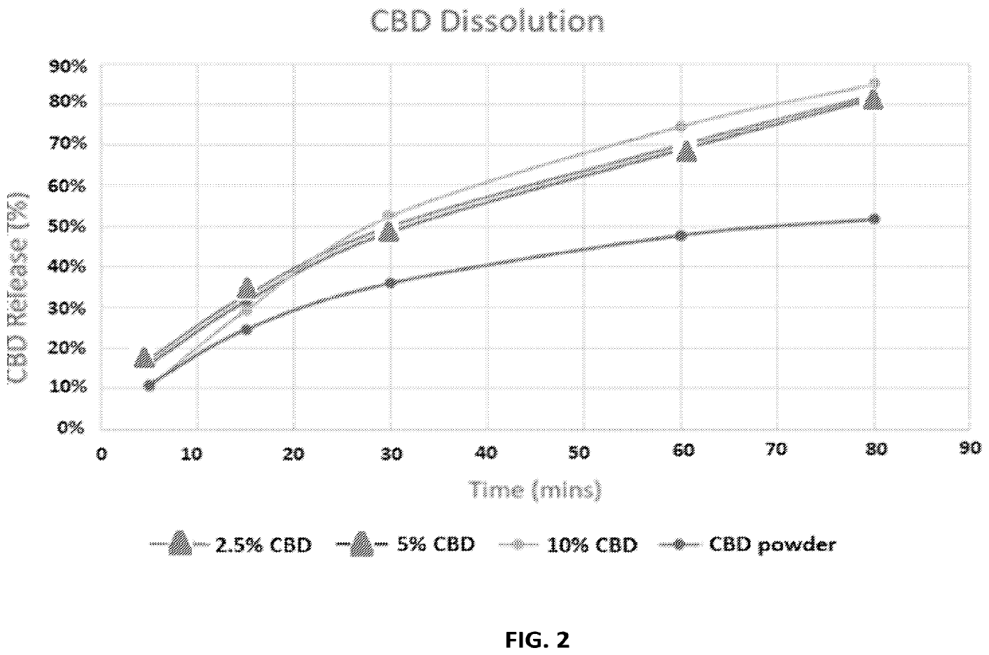

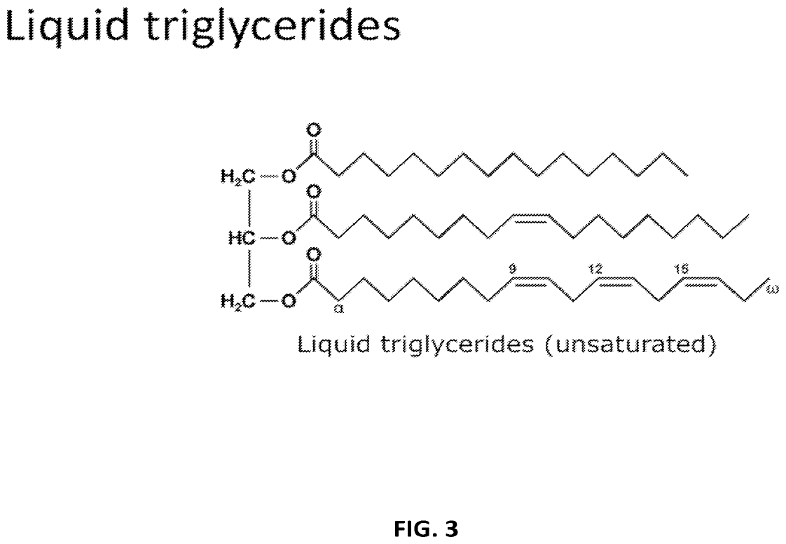

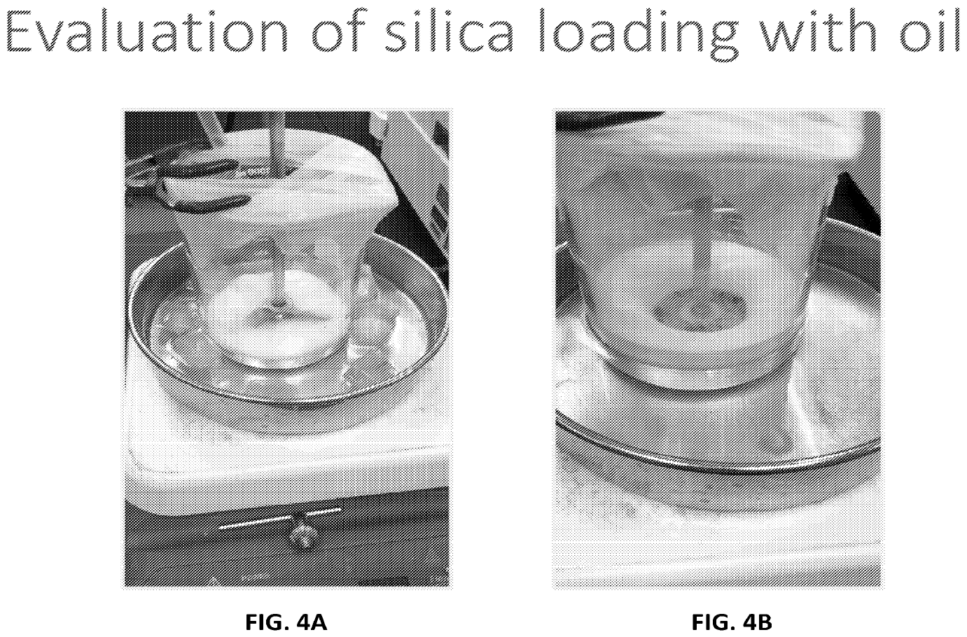

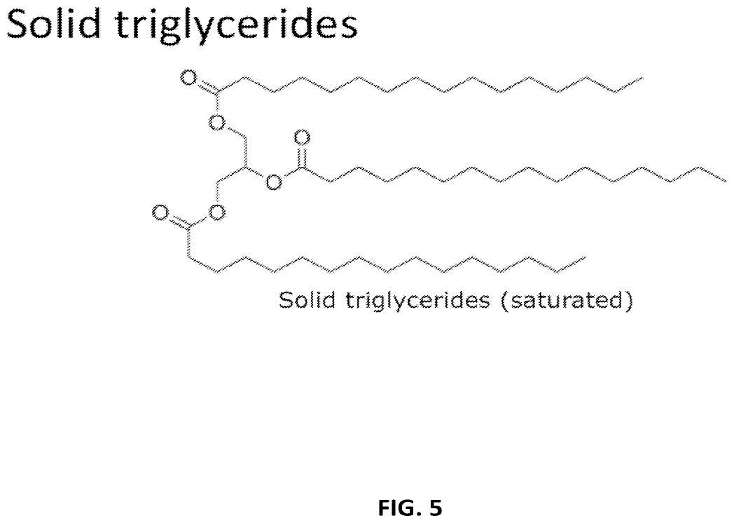

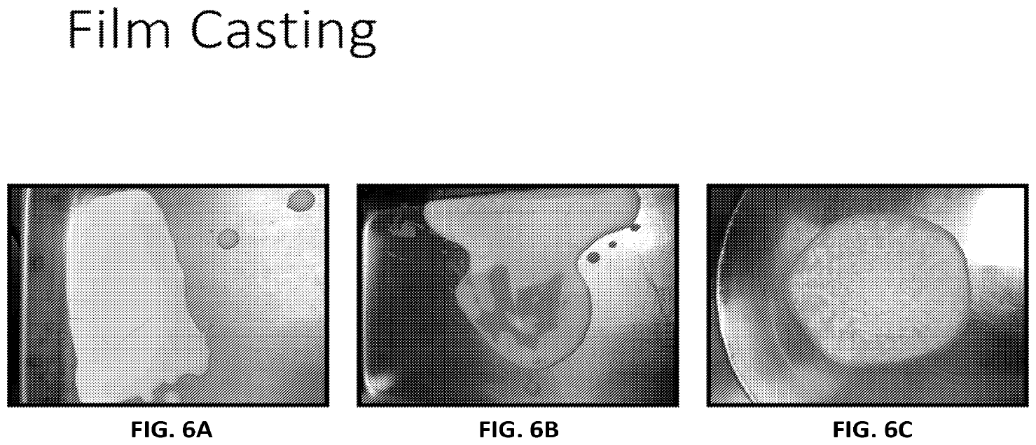

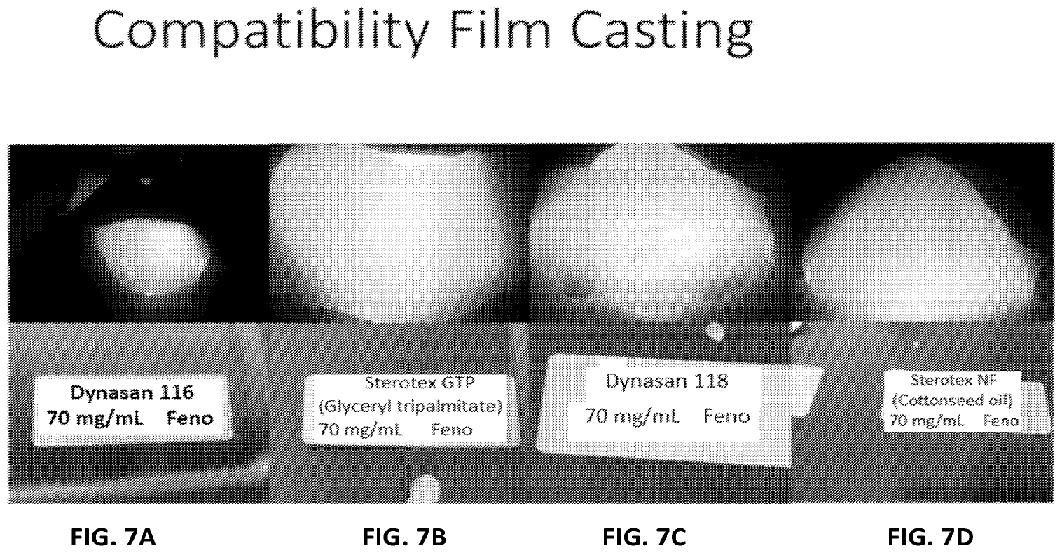

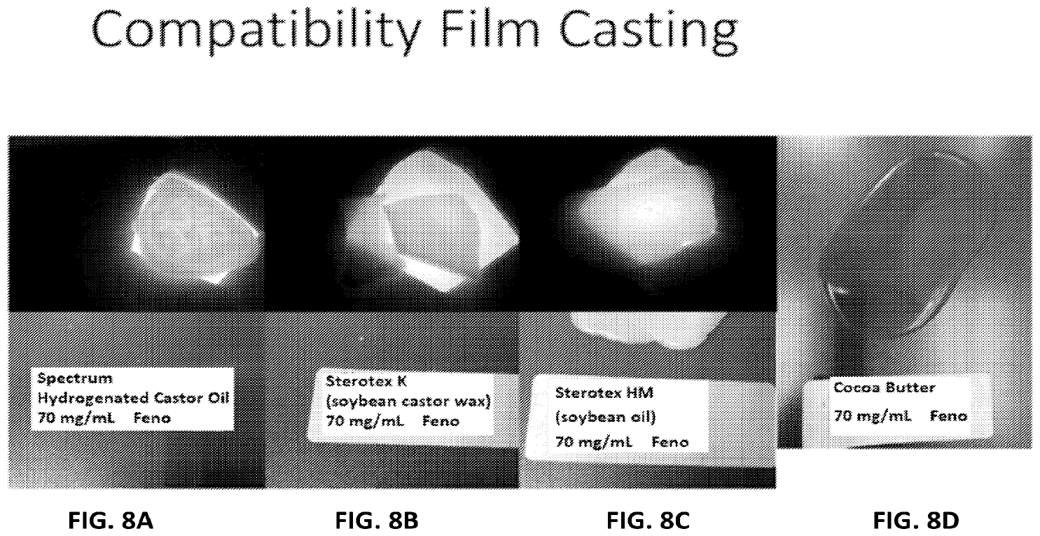

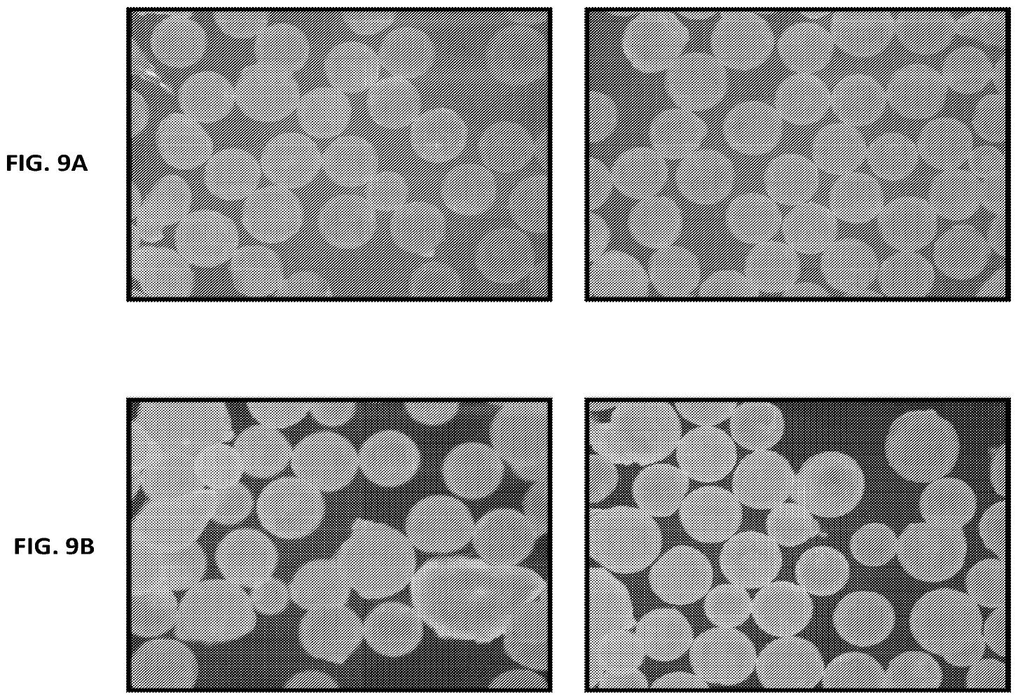

In one embodiment, the present disclosure further demonstrates the increased bioavailability of the formulation of the present disclosure with the dissolution profile as shown in FIGS. 1 and 2. The formulation of the present disclosure (Fenofibrate formulation) is compared with the placebo formulation.

Dissolution Media Conditions

The dissolution media is a 6.8 Phosphate buffer with 2% Tween 80 The media used to observe both cannabidiol and fenofibrate release using the USP Apparatus 2, 1 L per vessel, 37° C. and at 50 rpm. The solubility of actives studied was shown to be above amounts within this dissolution media.

Emulsion Formation and Dissolution Profile:

The formulation disintegration was tested using a placebo formulation or Fenofibrate at 5, 7.5, and 10%, Each mixture was placed into dissolution buffer mixed at 150 RPM and a temperature of 37° C. Each mixture was observed to have fully dispersed into the dissolution buffer between 10-15 minutes to form emulsions. After 60 minutes, the mixture can be poured through a 20 μm screen, leaving no visible residue.

FIGS. 1 and 2 further compare the dissolution profile of fenofibrate and cannabidiol (CBD) formulation, respectively, prepared according to the present disclosure vis-A-vis the raw powder of fenofibrate and cannabidiol.

Stability Data:

The pellets of example 1 were maintained in open conditions and exposed to a temperature of 23 (C and 30% RH for 30 days. There were no detectable impurities related to oxidation or photo degradation when analysed using a UPLC method.

### Example 2—Loading of Silica with Oil

Silica loading depends on mixing energy, rate of oil addition, droplet size, and temperature. Silica loading using SYLOID® XDP 3150 was found to cap out at 1.6 mg/ml. Pilot testing for optimal oil loading for ZEOPHARM 5191, ZEOPHARM 600 and SYLOID® XDP 3150 demonstrated results of about 2 mg/ml, 3 mg/ml, and 1.4 mg/ml, respectively.

### Example 3—Formulating the Silica/Oil/API Intermediate into an Oral Dosage Form

API is loaded into oil at 50-65 mg/ml, depending on a variety of factors including storage temperature. Oil loading onto silica is about 1.6 ml/g using SYLOID® XDP 3150. One mg of silica carries a maximum load of about 80-104 μg of API. For an orally dissolving tablet (ODT) using the oil/silica/API intermediate product, PHARMABURST® 500 or Mannogem EZ may be used to formulate the ODT at not greater than 12% silica (e.g., a 1000 mg tablet has 120 mg silica and 9.6-12.48 mg of API). Based on this information, greater than 50% loading is likely possible.

To formulate an orally dispersible platform (ODP), PHARMASPERSE® may be used in combination with the oil/silica/API intermediate product. PHARMASPERSE® can carry a load of 50% or more of the silica intermediate and still maintain excellent organoleptics. A 1 g dose can deliver 0.5 g of 33% silica and 21.1-27.5 mg of drug. A 2 g dose can deliver 1 g of 33% silica and 42.2-54.9 mg of drug. Other excipients, such as magnesium stearate, may be used in the ODP formulation.

### Example 4—Formulating the Solid Triglyceride/API Spray-Congealed Beads into an Oral Dosage Form

For a 1 to 2 g total dose size, the oral dosage form may comprise about 2% flavor and about 98% solid oil. A density of about 1.05-2.11 ml per 1-2 g dose, and drug loading of at least about 52.5-105.5 mg per 1-2 g dose, are expected.

### Example 5—Formulation Work; Calculation on Silica and Solid Oil Loading

Background Calculation for Silica

Oil loading of drug 50-65 mg/ml (variance depends on storage temperature). Oil loading onto silica is 1.6 ml/g (3150 based calculations). 1 mg of silica carries a max of 80-104 μg. E.g.—1 mg×1.6 μl/mg×50 μg/μl=80 μg

Dosage Based Calculations

ODT—Silica. Pharmaburst 500 and Mannogem EZ give poor performance at greater than 12% loading. 1000 mg tablet has 120 mg of silica and 9.60-12.48 mg of drug

ODP—Silica. Pharmasphere can carry over a 50% load of silica and maintain great organoleptics. E.g. 1 g total dose gives over 0.5 g of 33% silica and 21.1-27.5 mg of drug (Silica calculation not on dry basis); 2 g total dose gives over 1 g of 33% silica and 42.2-54.9 mg of drug (Silica calculation not on dry basis).

ODP—Solid oil. Assuming pure hard fat ODP would be 1-2 g total dose size w/2% flavor and 98% Solid oil (980-1960 mg). Assuming density close to 0.93 g/ml=1.05-2.11 ml per 1-2 g total dose. Upper limit of loading is currently in development. IP assessment is currently being done.

### Example 6—Formulation of Fenofibrate Loaded Beads

Beads comprising about 20%, about 30%, or about 40% fenofibrate were prepared according to the following formulas:

20% Fenofibrate (Feno)

| Product | mg | |
|---|---|---|
| Imwitor 900K | 2290 | 22.9% |
| Dynasan 116 | 3460 | 34.6% |
| Lecithin | 1170 | 11.7% |
| Kolliphor EL | 1100 | 11.0% |
| Fenofibrate | 2000 | 20.0% |
| total | 10000 | 100.2% |

30% Fenofibrate (Feno)

| Product | mg | |
|---|---|---|
| Imwitor 900K | 1990 | 19.9% |
| Dynasan 116 | 3010 | 30.1% |
| Lecithin | 1020 | 10.2% |
| Kolliphor EL | 980 | 9.8% |
| Fenofibrate | 3000 | 30.0% |
| total | 10000 | 100.0% |

40% Fenofibrate (Feno)

| Product | mg | |
|---|---|---|
| Imwitor 900K | 1710 | 17.1% |
| Dynasan 116 | 2590 | 25.9% |
| Lecithin | 870 | 8.7% |
| Kolliphor EL | 830 | 8.3% |
| Fenofibrate | 4000 | 40.0% |
| total | 10000 | 100.0% |

Briefly, molten lipid is sprayed into a cooling chamber and on contact with the cool air, congeals into spherical solid particles. The parameters to be considered when preparing a composition according to the present disclosure are the melting point of the excipients, the viscosity of the formulation and the cooling air temperature inside the chamber to allow instant solidification of the droplets. In this application, the spray coagulation method is used to prepare the solid lipid particles. The method is as described below:
a. Glyceryl monostearate (Imwitor 900K), glycerol tripalmitate (Dynasan 116) and polyethoxylated castor oil (Kolliphor EL) are co-melted in 50 ml beaker at 140° C. until the melting process is completed.
b. Once fully melted, lecithin is added to the melt and stirred at 200 rpm.
c. The mixture is then transferred to a heated syringe and allowed to equilibrate to 100° C.
d. The solid lipid mixture is then spray congealed through a Buchi B-390.
e. The mixture is pumped into the heat block, which contains the vibratory atomizer.
f. The nozzle size is set at 80 μm opening and the vibratory atomizer frequency and amplitude are configured to achieve individual droplet separation.
g. Droplets are cooled through a residence time within a cylinder designed to cool ambient air to approximately −6° C.
h. Solid lipid particles are then collected at the bottom of this chamber at a particle size distribution (PSD) between 75-400 μm.

FIG. 1B provides a graph showing the release of fenofibrate over time for each of the formulations described herein. To prepare the ‘20%+Silica’ formulation, the 20% fenofibrate formula was prepared in duplicate and the second batch blended with silica (about 5% of bead weight of Syloid 244) for 5 mins in a V shell blender.

As shown in FIG. 1B, drug (e.g., fenofibrate) release rate is enhanced, as compared to fenofibrate, when formulated with one or more of a glyceryl monostearate (e.g., Imwitor 900K), a glycerol tripalmitate (e.g., Dynasan 116), a polyethoxylated castor oil e.g., (Kolliphor EL), a surfactant, and an emulsifier. Dissolution data generated in 900 ml of pH 6.8 phosphate buffer with 2% Tween 80.

1. A lipid particle comprising:
one or more poorly soluble active ingredients;
one or more lipids; and/or
one or more Hydrophile-Lipophile Balance (HLB) modifying agents to improve a bioavailability of the one or more poorly soluble active ingredients within a subject, wherein the total concentration of the one or more poorly soluble active ingredients in the particle is between about 20% and about 40% by weight.

2. The lipid particle of claim 1, wherein the one or more lipids is selected from triglycerides, monoglycerides, fatty acids, fluorinated lipids, neutral fats, phosphatides, oils, glycerol di-oleate, glycerol mono-oleate, tri-stearin, glycerol di-stearin, glycerol mono-stearin, tri-palmintin, glycerol di-palmate, glycerol mono-palmate, tri-myristin, glycerol di-myristate, glycerol mono-myristate, hydrogenated palm oil, fractionated palm oil, hydrogenated soybean oil, hydrogenated cottonseed oil, hydrogenated castor oil, and steroids.

3. The lipid particle of claim 2, wherein the one or more lipids comprises a triglyceride.

4. The lipid particle of claim 3, wherein the triglyceride comprises glycerol tripalmitate.

5. The lipid particle of claim 4, wherein the glycerol tripalmitate comprises Dynasan 116.

6. The lipid particle of any one of claims 2-5, wherein the one or more lipids comprises a monoglyceride.

7. The lipid particle of claim 6, wherein the monoglyceride is selected from monolaurin, glyceryl monostearate, and glycerol hydroxy stearate.

8. The lipid particle of claim 5, wherein the monoglyceride comprises glyceryl monostearate.

9. The lipid particle of claim 8, wherein the glycerol monostearate comprises Imwitor 900K.

10. The lipid particle of any one of claims 1-9, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnanzine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

11. The lipid particle of any one of claims 1-10, wherein the concentration of the one or more poorly soluble active ingredients in the particle is between about 30% and about 40% by weight.

12. The lipid particle of any one of claims 1-11, wherein the lipid particle is a solid lipid particle.

13. The lipid particle of any one of claims 1-12, comprising more than one lipid, wherein the total amount of lipid is between about 20% and 80% by weight.

14. The lipid particle of any one of claims 1-13, comprising between about 20% and 25% by weight of the one or more poorly soluble active ingredients, between about 18% and 28% by weight of glyceryl monostearate, and between about 30% and 40% by weight of glycerol tripalmitate.

15. The lipid particle of claim 14, comprising about 20% by weight of the one or more poorly soluble active ingredients, about 23% by weight of glyceryl monostearate, and about 35% of glycerol tripalmitate.

16. The lipid particle of any one of claims 1-13, comprising between about 27% to 32% by weight of the one or more poorly soluble active ingredients, between about 15% and 25% by weight of glyceryl monostearate, and between about 25% and 35% of glycerol tripalmitate.

17. The lipid particle of claim 16, comprising about 30% by weight of the one or more poorly soluble active ingredients, about 20% by weight of glyceryl monostearate, and about 30% of glycerol tripalmitate.

18. The lipid particle of any one of claims 1-13, comprising between about 35% and 40% by weight of the one or more poorly soluble active ingredients, between about 12% and 22% by weight of glyceryl monostearate, and between about 21% and 31% of glycerol tripalmitate.

19. The lipid particle of claim 18, comprising about 40% by weight of the one or more poorly soluble active ingredients, about 17% by weight of glyceryl monostearate, and about 26% of glycerol tripalmitate.

20. The lipid particle of any one of claims 1-19, wherein the one or more Hydrophile-Lipophile Balance (HLB) modifying agents are selected from the group consisting of a surfactant, an emulsifier, lecithin, polyethoxylated castor oil, and Kolliphor EL.

21. The lipid particle of any one of claims 1-19, wherein the one or more Hydrophile-Lipophile Balance (HLB) modifying agents comprise lecithin and polyethoxylated castor oil.

22. The lipid particle of claim 20 or claim 21, comprising between about 20% and 25% by weight of the one or more poorly soluble active ingredients, between about 7% and 17% by weight of lecithin, and between about 6% and 16% by weight of polyethoxylated castor oil.

23. The lipid particle of claim 22, comprising about 20% by weight of the one or more poorly soluble active ingredients, about 12% by weight of lecithin, and about 11% by weight of polyethoxylated castor oil.

24. The lipid particle of claim 20 or claim 21, comprising between about 27% and 32% by weight of the one or more poorly soluble active ingredients, between about 5% and 15% by weight of lecithin, and between about 5% and 15% by weight of polyethoxylated castor oil.

25. The lipid particle of claim 24, comprising about 30% by weight of the one or more poorly soluble active ingredients, about 10% by weight of lecithin, and about 10% by weight of polyethoxylated castor oil.

26. The lipid particle of claim 20 or claim 21, comprising between about 35% and 40% by weight of the one or more poorly soluble active ingredients, between about 4% and 14% by weight of lecithin, and between about 3% and 13% by weight of polyethoxylated castor oil.

27. The lipid particle of claim 26, comprising about 40% by weight of the one or more poorly soluble active ingredients, about 9% by weight of lecithin, and about 8% by weight of polyethoxylated castor oil.

28. The lipid particle of any one of claims 21-27, wherein the polyethoxylated castor oil comprises Kolliphor EL.

29. The lipid particle of any one of claims 1-28, wherein the one or more poorly soluble active ingredients comprise fenofibrate.

30. The lipid particle of any one of claims 1-29, wherein the one or more poorly soluble active ingredients comprise cannabidiol.

31. The lipid particle of any one of claims 1-30, further comprising silica.

32. The lipid particle of any one of claims 1-31, wherein a plurality of the lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm; or wherein a plurality of the lipid particles have a size distribution of from about 10 μm to about 500 μm, about 20 μm to about 475 μm, about 30 μm to about 450 μm, about 40 μm to about 425 μm, about 50 μm to about 400 μm, about 60 μm to about 375 μm, about 70 μm to about 350 μm, about 80 μm to about 325 μm, about 90 μm to about 325 μm, or about 100 μm to about 300 μm.

33. The lipid particle of any one of claims 1-32, wherein the particle has a circularity greater than about 0.90.

34. The lipid particle of any one of claims 1-33, wherein the particle has a circularity greater than about 0.95.

35. The lipid particle of any one of claims 1-34, wherein the particle has an aspect ratio greater than about 0.90.

36. The lipid particle of any one of claims 1-35, wherein the particle has an aspect ratio greater than about 0.95.

37. A method of manufacturing a solid lipid particle, wherein the method comprises spray coagulating a lipid particle composition comprising:
one or more poorly soluble active ingredients,
one or more lipids, and/or
one or more Hydrophile-Lipophile Balance (HLB) modifying agents to improve a bioavailability of the one or more poorly soluble active ingredients within a subject.

38. The method of claim 37, wherein the solid lipid particle composition further comprises silica.

39. The method of claim 37, comprising the steps of:
a) co-melting a first lipid, a second lipid, and a first HLB modifying agent, thereby producing a melt;
b) adding a second HLB modifying agent to the melt;
c) adding one or more of the poorly soluble active ingredients to the melt;
d) co-melting the melt with the second HLB modifying agent and the one or more poorly soluble active ingredients, thereby preparing a mixture;
e) transferring the mixture to a heated vessel;
f) equilibrating the mixture with the heated vessel;
g) spray coagulating the mixture through a spraying chamber to achieve individual droplet separation; and
h) cooling the droplets to form solid lipid particles.

40. The method of claim 39, wherein c) further comprises adding silica to the melt and the mixture of d) further comprises unmelted silica.

41. The method of claim 39 or claim 40, further comprising i) coating or co-melting the solid lipid particles with a polymer.

42. The method of any one of claims 39-41, further comprising j) preparing a solid suspension of the solid lipid particles with a material having a high melting point.

43. The method of any one of claims 39-42, wherein the first lipid is glyceryl monostearate and the second lipid is glycerol tripalmitate.

44. The method of claim 43, wherein the glyceryl monostearate is Imwitor 900K and the glycerol tripalmitate is Dynasan 116.

45. The method of any one of claims 39-44, wherein the first HLB modifying agent is a polyethoxylated castor oil and the second HLB modifying agent is lecithin.

46. The method of claim 45, wherein the polyethoxylated castor oil is Kolliphor EL.

47. The method of any one of claims 37-46, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnanzine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

48. The method of any one of claims 37-47, wherein the one or more poorly soluble active ingredients comprise fenofibrate, cannabidiol, or a combination thereof.

49. The method of any one of claims 37-48, wherein the total concentration of the one or more poorly soluble active ingredients in the solid lipid particle is between about 20% and about 40% by weight.

50. The method of any one of claims 37-49, wherein the solid lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm.

51. The method of any one of claims 37-50, wherein the solid lipid particle has a circularity greater than about 0.90.

52. The method of any one of claims 37-51, wherein the solid lipid particle has a circularity greater than about 0.95.

53. The method of any one of claims 37-52, wherein the solid lipid particle has an aspect ratio greater than about 0.90.

54. The method of any one of claims 37-53, wherein the solid lipid particle has an aspect ratio greater than about 0.95.

55. A method of manufacturing a solid lipid particle, wherein the method comprises hot melting a lipid particle composition comprising:
one or more poorly soluble active ingredients,
one or more lipids, and/or
one or more Hydrophile-Lipophile Balance (HLB) modifying agents to improve a bioavailability of the one or more poorly soluble active ingredients within a subject.

56. The method of claim 55, wherein the hot melting comprises one or both of hot melt extrusion and hot melt granulation.

57. The method of claim 55 or claim 56, wherein the one or more lipids are selected from glyceryl monostearate and glycerol tripalmitate.

58. The method of claim 57, wherein the glyceryl monostearate is Imwitor 900K and the glycerol tripalmitate is Dynasan 116.

59. The method of any one of claims 55-58, wherein the lipid particle composition further comprises silica.

60. The method of any one of claims 55-59, wherein the one or more HLB modifying agents are selected from polyethoxylated castor oil and lecithin.

61. The method of claim 60, wherein the polyethoxylated castor oil is Kolliphor EL.

62. The method of any one of claims 55-61, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

63. The method of any one of claims 55-62, wherein the one or more poorly soluble active ingredients comprise fenofibrate, cannabidiol, or a combination thereof.

64. The method of any one of claims 55-63, wherein the total concentration of the one or more poorly soluble active ingredients in the solid lipid particle is between about 20% and about 40% by weight.

65. The method of any one of claims 55-64, wherein the solid lipid particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 μm, or about 75 μm to about 400 μm.

66. The method of any one of claims 55-65, wherein the solid lipid particle has a circularity greater than about 0.90.

67. The method of any one of claims 55-66, wherein the solid lipid particle has a circularity greater than about 0.95.

68. The method of any one of claims 55-67, wherein the solid lipid particle has an aspect ratio greater than about 0.90.

69. The method of any one of claims 55-68, wherein the solid lipid particle has an aspect ratio greater than about 0.95.

70. A method of manufacturing a solid lipid particle mixture, the method comprising:
a) co-melting a first lipid, a second lipid, and a first Hydrophile-Lipophile Balance (HLB) modifying agent, thereby producing a melt;
b) adding a second HLB modifying agent to the melt,
c) adding one or more poorly soluble active ingredients to the melt,
d) co-melting the melt with the second HLB modifying agent and the one or more poorly soluble active ingredients, thereby preparing a mixture,
e) film casting the mixture, the film casting comprising pouring the mixture and cooling the mixture until hardened, thereby producing the solid lipid particle mixture.

71. The method of claim 70, wherein c) further comprises adding silica to the melt and the mixture of d) further comprises unmelted silica.

72. The method of claim 70 or claim 71, further comprising f) grinding the solid lipid particle mixture into a lipid powder.

73. The method of claim 72, further comprising g) coating or co-melting the lipid powder with a polymer.

74. The method of claim 72 or claim 73, further comprising h) preparing a solid suspension of the lipid powder with a material having a high melting point.

75. The method of any one of claims 70-74, wherein the first lipid is glyceryl monostearate and the second lipid is glycerol tripalmitate.

76. The method of claim 75, wherein the glyceryl monostearate is Imwitor 900K and the glycerol tripalmitate is Dynasan 116.

77. The method of any one of claims 70-76, wherein the first HLB modifying agent is a polyethoxylated castor oil and the second HLB modifying agent is lecithin.

78. The method of claim 77, wherein the polyethoxylated castor oil is Kolliphor EL.

79. The method of any one of claims 70-78, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnanzine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

80. The method of any one of claims 70-79, wherein the one or more poorly soluble active ingredients comprise fenofibrate, cannabidiol, or a combination thereof.

81. The method of claim 72 or claim 73 wherein the lipid powder comprises solid lipid particles.

82. The method of claim 81, wherein the total concentration of the one or more poorly soluble active ingredients in the solid lipid particle is between about 20% and about 40% by weight.

83. The method of claim 81 or claim 82, wherein the solid lipid particles have a size distribution of from about 10 μm to about 500 μm, about 20 μm to about 475 μm, about 30 μm to about 450 μm, about 40 μm to about 425 μm, about 50 μm to about 400 μm, about 60 μm to about 375 μm, about 70 μm to about 350 μm, about 80 μm to about 325 μm, about 90 μm to about 325 μm, or about 100 μm to about 300 μm.

84. The method of any one of claims 81-83, wherein the solid lipid particle has a circularity greater than about 0.90.

85. The method of any one of claims 81-84, wherein the solid lipid particle has a circularity greater than about 0.95.

86. The method of any one of claims 81-85, wherein the solid lipid particle has an aspect ratio greater than about 0.90.

87. The method of any one of claims 81-86, wherein the solid lipid particle has an aspect ratio greater than about 0.95.

88. A solid dosage form for oral delivery comprising:
a) a poorly soluble active ingredient; and
b) a solid triglyceride;
wherein the poorly soluble active ingredient and the solid triglyceride are spray congealed to form a plurality of beads.

89. A solid dosage form for oral delivery comprising a plurality of spray congealed beads, the beads comprising:
a) a poorly soluble active ingredient; and
b) a solid triglyceride.

90. The solid dosage form of claim 88 or claim 89, wherein the solid triglyceride is selected from DYNASAN® 116, DYNASAN® 118, STEROTEX® GTP, STEROTEX® NF, STEROTEX® K, hydrogenated castor oil, and cocoa butter.

91. The solid dosage form of any one of claims 88-90, wherein the beads are combined with one or more excipients to form the solid dosage form.

92. A solid dosage form for oral delivery, comprising:
a) a poorly soluble active ingredient;
b) a liquid triglyceride; and
c) silica
wherein the poorly soluble active ingredient, the liquid triglyceride, and silica are mixed to form a homogenous mixture.

93. The solid dosage form of claim 92, wherein the liquid triglyceride is selected from sesame oil, olive oil, palm oil, cottonseed oil, corn oil, rapeseed oil, and safflower oil.

94. The solid dosage form of claim 92 or claim 93, wherein the silica is selected from SYLOID® XDP 3150, SYLOID® XDP 3050, ZEOPHARM™ 5191, and ZEOPHARM™ 600.

95. The solid dosage form of any one of claims 92-94, wherein the homogenous mixture is combined with one or more excipients to form the solid dosage form.

96. The solid dosage form of any one of claims 88-95, wherein the active ingredient is present in an amount of about 5 mg/mL to about 100 mg/mL, an amount of about 10 mg/mL to about 75 mg/mL, an amount of about 50 mg/mL, or an amount of about 70 mg/mL.

97. The solid dosage form of any one of claims 88-96, wherein the one or more poorly soluble active ingredients is selected from amiodarone, atorvastatin, azithromycin, carbamazepine, carvedilol, cisapride, cyclosporine, danazol, dapsone, fenofibrate, cannabidiol, gliclazide, glyburide, glimepiride, glipizide, indinavir, itraconazole, ketoconazole, lansoprazole, lovastatin, repaglinide, pioglitazone, progesterone, ritonavir, rosiglitazone, saquinavir, sirolimus, tacrolimus, tamoxifen, praziquantel, diclofenac, ibuprofen, co enzyme q10, paclitaxel, glibenclamide, penclomedine, halofantrine, cyclosporin, atorvaquone, ezitimibe, cinnarizine, oxyresveratrol, lopinavir, darunavir, olmesarton medoxamil, puerarin, lutein, isradipine, lornoxicam, docetaxel, flurbiprofen, ciprofloxacin, furosemide, clopidogrel, dutasteride, amprenavir, saquinavir, calcitrol, valproic acid, isotretinoin, dronabinol, clofazimine, bexarotene, doxecalciferol, sirolimus, dutasteride, tipranavir, paricalcitrol, topotecen, loratadine, nintedanib, calcifediol, and combinations thereof.

98. The solid dosage form of any one of claims 88-97, wherein the one or more poorly soluble active ingredients comprise fenofibrate, cannabidiol, or a combination thereof.

99. The solid dosage form of any one of claims 88-91, wherein the beads are lipid particles.

100. The solid dosage form of claim 99, wherein the lipid particles are solid lipid particles.

101. The solid dosage form of any one of claims 92-95, wherein the homogenous mixture comprises particles comprising silica loaded with the liquid triglyceride and the poorly soluble active ingredient.

102. The solid dosage form of claim 95, wherein the one or more excipients are blended with particles to form the homogenous mixture, the particles comprising silica loaded with the liquid triglyceride and the poorly soluble active ingredient.

103. The solid dosage form of claim 101 or claim 102, wherein the particles are lipid particles.

104. The solid dosage form of any one of claims 99-103, wherein the total concentration of the one or more poorly soluble active ingredients in the particles is between about 20% and about 40% by weight.

105. The solid dosage form of any one of claims 99-104, wherein the particles have a size distribution of from about 10 μm to about 500 μm, about 20 μm to about 490 μm, about 30 μm to about 480 μm, about 40 μm to about 470 μm, about 50 μm to about 460 μm, about 50 μm to about 450 μm, about 60 μm to about 440 μm, about 60 μm to about 430 μm, about 70 μm to about 420 μm, about 70 μm to about 410 m, or about 75 μm to about 400 μm; or wherein the particles have a size distribution of from about 10 μm to about 500 μm, about 20 μm to about 475 μm, about 30 μm to about 450 m, about 40 μm to about 425 m, about 50 μm to about 400 μm, about 60 μm to about 375 m, about 70 μm to about 350 m, about 80 μm to about 325 μm, about 90 μm to about 325 m, or about 100 μm to about 300 μm.

106. The solid dosage form of any one of claims 99-105, wherein the particles have a circularity greater than about 0.90.

107. The solid dosage form of any one of claims 99-106, wherein the particles have a circularity greater than about 0.95.

108. The solid dosage form of any one of claims 99-107, wherein the particles have an aspect ratio greater than about 0.90.

109. The solid dosage form of any one of claims 99-108, wherein the particles have an aspect ratio greater than about 0.95.
